(19) United States
(12) Patent Application Publication
(10) Pub. No.: US 2004/0247567 A1
(43) Pub. Date: Dec. 9, 2004

(54) Microvascular free flaps for local or systemic delivery

(76) Inventor: Geoffrey C. Gurtner, New York, NY (US)

Correspondence Address: JONES DAY, 222 EAST 41ST ST, NEW YORK, NY, 10017, US

(21) Appl. No.: 10/477,433

(22) PCT Filed: May 7, 2002
(86) PCT No.: PCT/US02/14483

Related U.S. Application Data
(60) Provisional application No. 60/289,452, filed on May 7, 2001.

(51) Int. Cl. A61K 48/00; A61K 9/127

(52) U.S. Cl. 424/93.2; 424/450

(57) ABSTRACT

The present invention relates to methods of ex-vivo modification of mammalian tissue, via genetic transformation or introduction of cells, followed by implantation of the modified tissue into a patient in need thereof. Preferably, the tissue is microvascular free flap (or microvascular bed) tissue. A tissue explant is detached from the native circulation of a donor, transfected ex vivo, and then attached (anastomosed) to a recipient, either the donor or another patient. In a preferred embodiment, the mammalian tissue is human tissue and the patient is a human patient. Transfection with a nucleic acid encoding a product of interest is performed by contacting the selected tissue with a vector, preferably a viral vector, most preferably an adenoviral vector, that comprises the nucleic acid encoding the product of interest. The nucleic acid encoding the product of interest is driven by regulatory element such as an inducible, constitutive or cell-specific promoter, preferably an inducible or constitutive promoter. After genetic transformation of the selected tissue, the tissue is flushed to remove the vector not incorporated into the cells of the tissue. The tissue is then attached to the native circulation of the recipient using microvascular techniques. In one aspect, the invention provides methods of local delivery of a product (protein) of interest. In another aspect, the invention provides methods of systemic delivery of a product of interest. In yet another aspect, the invention provides methods of both local and systemic delivery of a product of interest. In yet another aspect, the invention provides methods for producing a “neo-organ,” i.e., a non-naturally occurring vascularized tissue that provides a function of a gland or organ, or that supplements the function of a gland or organ, and that delivers locally or systemically a product of interest to a patient in need thereof.

This application claims priority benefits of application No. 60/289,452 filed May 7, 2001, the entire disclosure of which is incorporated herein by reference in its entirety.

1. TECHNICAL FIELD

The present invention relates to methods of ex-vivo modification of vertebrate tissue, via genetic transformation or introduction of cells, for local or systemic delivery of a therapeutic product of interest. The present invention also relates to methods for producing a “neo-organ,” i.e., a non-naturally occurring vascularized tissue that provides a function of a gland or organ, or that supplements the function of a gland or organ.

### 2. BACKGROUND OF THE INVENTION

Gene therapy using viral vectors holds great clinical promise but has been limited by difficulties in developing targeted, high-level gene expression with acceptable host toxicity. Human gene therapy has been a disappointment in clinical trials and in some gene therapy trials serious concerns regarding safety have been raised (see, e.g., Marshall, 1999, Science 286: 2244-45).

Three major obstacles prevent gene therapy from achieving clinical utility: 1) the difficulty in obtaining adequate, sustained gene expression in vivo, 2) the difficulty in localizing gene expression to areas of clinical interest and 3) the difficulty in avoiding the systemic immune response and resulting host toxicity (Anderson, 1999, New York Times 44; Somia et al., 2000, Nature Reviews Genetics 1: 91-99; Glorioso et al., 2001, Nat. Med. 7: 3340; Li et al., 2000, Gene Ther. 7: 31-34; Strayer, 1999, Expert Opin. Investig. Drugs 8: 2159-72). Currently, human gene therapy requires systemic gene administration (in vivo) or removal of isolated cells for modification by transfection or infection with vectors carrying recombinant genes (ex vivo), followed by subsequent re-infusion or re-implantion into the body (“autologous cell transfer”).

Sharked et al. and Qin et al. disclose genetically modifying an organ such as a liver or heart ex vivo prior to transplantation (Sharked et al., 1994, Transplantation 57(10): 1508-11; Qin etal., 1995, Transplantation 59(6): 809-16).

Taub describes modification of microvascular flaps in situ in an animal with an angiogenic gene to improve vascularity and enhance flap survival (Taub et al., 1998, Plast. Recon. Surg. 102(6): 2033-9). The drawback of this approach, however, is that the flap is genetically modified within the animal, which has the drawbacks of exposing the animal to a potential systemic immune response and host toxicity.

New approaches to gene therapy are needed that could possibly circumvent these difficulties. Such a method would represent an advance over currently utilized techniques of gene delivery and would be an ideal method for targeted gene transfer in patients undergoing microvascular flap transfers following surgery, in particular, oncologic surgery. The present invention provides such an approach.

2.1. Therapeutic Regimens for Head and Neck Cancers

Patients with recurrent or locally metastatic head and neck cancers present unique challenges to the head and neck surgeon. Head and neck tumors are characterized by a significant degree of morbidity and mortality caused in large part by local tumor extension and invasion. One particularly aggressive and common form of head and neck tumor is head and neck squamous cell carcinoma (SCC). SCC tumors, accounting for 6% of all new cancers in this country and 12,500 deaths each year (Landis et al., 1998, Cancer J. Clin., 1:6-29), are particularly difficult to obtain local control following surgery. The head and neck surgeon is frequently involved in the care of these patients, often in combination with the reconstructive plastic surgeon. This inter-disciplinary care has resulted in advancements in surgical ablative techniques as well as the availability of novel reconstructive modalities. However, despite more aggressive surgery (made possible in part by the availability of microsurgical reconstruction) as well as novel radiologic and chemotherapeutic approaches, the mortality rates for this heterogenous population of tumors have not significantly improved during the last 30 years (Vokes et al., 1993, N. Engl. J. Med. 328: 184-191). This disappointing reality highlights the need for novel therapeutic approaches for head and neck SCC.

The anatomic complexity in the head and neck region limits the ability to obtain local control, with increased surgical margins translating to decreased quality of life. For this reason, much effort has been expended on developing anti-tumor gene therapy. Gene therapy is currently being explored as a therapeutic treatment for a variety of malignancies, including those of the head and neck (Ganly et al. 2000, Eur. J. Surg. Onc. 26: 338-343). It can deliver either immunomodulatory or cytotoxic genes that can target tumor cells. The number of different molecular targets that can be used for gene therapy is enormous; some that have been studied for malignancies, including those of the head and neck, include p53, interferon-beta, cyclin inhibitors, IL-2, IL-4, IL-12 and HLA-B7 (Breau et al., 1996, Curr. Opin. Oncol. 8: 227-31; Li et al., 1999, Clin. Cancer Res. 5(6): 1551-6; reviewed in Ganly et al., 2000, Eur. J. Surg. Onc. 26: 338-343; Gleich, 2000, Laryngoscope 110: 708-726). Unfortunately, gene therapy currently has drawbacks that prevent it from achieving clinical utility. Systemic administration is limited by toxic side effects and attempts at local therapy via injections or topical application have proven cumbersome and impractical (Udvardi et al., 1999, Jour. Mol. Med. 77(10): 744-50).

The advent of reconstructive microsurgery, however, has greatly aided the care of the oncologic head and neck patient. Free tissue transfer is now routinely used to close defects that were not amenable to closure several decades ago, and has improved the care of the head and neck patient by enabling improved surgical palliation, such as adequate oral continence following removal of a tumor of the mouth. In addition, wider resections are now routinely carried out due to the availability of reliable reconstructive options.

While free tissue transfers have mostly been used for closure of defects (i.e., as fillers) and to enable some return of function (e.g., in the restoration of a competent oral sphincter or space esophageal tube), no methods are currently available that exploit their potential for gene therapy. The present invention provides such methods.

2.2. Tissue Engineering and Replacement Organs

A major obstacle preventing the development of techniques to engineer replacements for failing organs is the inability to adequately vascularize tissues created in vitro. Intact organs contain a highly complex three-dimensional network of arterioles, capillaries and venules, which allow for the efficient exchange of oxygen, nutrients and metabolic intermediaries. Tissue engineering approaches using the implantation of cells onto resorbable matrices have had success in replicating simple, relatively avascular structures such as cartilage or bone, but have not been able to create more complex organs (Stock et al., 2001, Annu. Rev. Med. 52: 443-51; Kaihara et al., 1999, Arch. Surg. 134: 1184-88). Similarly, stem cell technology holds tremendous promise to “patch” or “regenerate” partially damaged organs in vivo, but it is difficult to envision the creation of new organs in vitro using current methodologies.

Creating a functional organ in vitro generally requires two elements: sufficient cellular mass for physiologic effect and organization of this mass into a three-dimensional structure able to be reintegrated into the systemic circulation. Conceptually, the problem with both tissue engineering and stem cell strategies is that they are cell-based approaches to an organ-level problem. The original goal for tissue engineering was to create biological substitutes to restore, maintain or improve tissue function (Langer et al., 1993, Science 260: 920-26). However the classic tissue engineering paradigm using biodegradable matrices seeded with cells and vascularized by wound angiogenesis has been unsuccessful in situations where cellular metabolic needs cannot be met by simple difflusion (i.e., large complex organs).

A degree of success has been achieved in maintaining cell constructs in vivo but the limiting factor is the blood supply for tissue-engineered structures (Kaihara et al., 1999, Arch. Surg. 134: 1184-88). In addition, the matrix itself has presented entirely new problems related to the introduction of foreign material, which often results in host rejection of the construct (Mikos et al., 1998, Drug Deliv. Rev. 33: 111-139). Furthermore, it is unclear how a tissue engineered construct would be integrated into the vasculature of a patient. Until these problems are resolved, even the most optimized results cannot transform the field of tissue engineering from a technique supplying isolated cells to a technique that truly generates tissue to replace lost or defective organs.

Tremendous interest has been generated in utilizing adult or embryonic stem cells for organ regeneration. Stem cells appear to possess the ability to replicate and differentiate into the broad-spectrum of cells existing in complex organs (Pittenger et al., 1999, Science 284: 143-7). Adult stem cells are able to repopulate and regenerate partially injured organs in experimental animal models (Bianco et al., 2001, Nature: 414: 118-21; Asahara et al., 2000, Gene Ther. 7: 451 -7; Baldwin 1999, Curr. Opin. Pediatr. 11:,413-8). However, this in vivo regeneration always takes place within the pre-existing template of an existing organ. It is unknown whether, or how, embryonic stem cells could be manipulated ex vivo to replace an absent or failing organ. Embryonic stem cells clearly have the ability to guide the three-dimensional patterning of organs within the specialized environment of the embryo (Baldwin 1999, Curr. Opin. Pediatr. 11: 413-8). It is difficult at present, however, to envision how this plasticity could be exploited post-natally to create new organs. The most plausible scenarios involve partial or total human cloning (Tian et al., 2001,Trends Cardiovasc. Med. 11: 313-7) which raise a host of philosophical and ethical questions that may never be resolved. We lack sufficient understanding of the principles governing normal organ development and physiology necessary to predictably manipulate in vitro the pluripotentiality of stem cells for therapeutic purposes.

Recent discoveries in developmental biology implicate the vasculature as a key determinant for both organ development and physiologic function. There is now clear evidence that embryologic development of the vasculature and organogenesis are interdependent. For example, the interaction of nascent hepatic cells with angioblasts or endothelial cells, prior to blood vessel formation, is essential for the earliest stages of liver bud outgrowth to proceed (Matsumoto et al., 2001, Science 294: 559-63). In a separate study, the differentiation of insulin-producing pancreatic cells from the foregut endoderm was shown to depend on endothelial-endoderm interactions (Lammert et al., 2001, Science 294: 564-7). Since the vasculature plays a critical early role as an inductive element for organ cell differentiation and proliferation, the successful and early incorporation of a vascular network into engineered tissues would likewise appear to be essential.

Ideally, the best strategy for creating vascularized tissue engineered constructs is to fully understand and manipulate the processes active during developmental vascular patterning. Tremendous progress has been made in understanding the mechanisms governing blood vessel growth during both development and in the adult. The vascular system develops through a combination of vasculogenesis (de novo vessel formation from bone marrow derived precursors) and angiogenesis (migration and proliferation of differentiated endothelial cells from preexisting vessels) (Risau et al., 1988, Development 102: 471-8). Factors such as VEGF, PDGF, TGF-β, b-FGF, the angiopoietins and ephrin isoforms, are critical to neovascularization in both the embryo and adult, and the mechanisms of their actions are areas of active research (Fong et al., 1995, Nature 376: 66-70; Sato et al., 1995, Nature 376: 70-4; Shalaby et al., 1995, Nature 376: 62-6; Folkinan et al., 1996, Cell 87: 1153-5; Yancopoulos et al., 2000, Nature 407: 242-8).

Although we have made significant progress in the identification of growth factors important for blood vessel formation, our understanding of the genetic mechanisms underlying vascular patterning remains woefully primitive. The recent demonstration of the specific expression of ephrin-B2 by arteries and ephrin-B4 by veins (Wang et al., 1998, Cell 93: 741-53; Adams et al., 1999, Genes Dev.13: 295-306) as well as notch expression in arterial vessels (Lawson et al., 2001, Development 128: 3675-83;Villa et al., 2001, Mech. Dev.108: 161-4), provides but two examples of the myriad genetic differences underlying vessel subtypes and underscores the inherent difficulties in coaxing the growth of a functional vascular network in vitro. It is apparent that genetic engineering of a large organ requiring a functional microcirculation may be an extraordinarily difficult task to accomplish de novo.

Within the surgical literature, it has been known for at least thirty years that humans (as well as other mammals) possess self-contained expendable microvascular beds (Armstrong et al., 2001, Clin. Plast. Surg. 28: 671-86; Buncke et al., 1996, Plast. Reconstr. Surg. 98: 1122-3). Examples in humans include the omentum, the temporoparietal fascia, and the transverse rectus abdominis myocutaneous tissue, among hundreds of others (Lieberrnann et al., 1991, Neth. J. Surg. 43: 136-44; Brent et al., 1985, Plast. Reconstr. Surg. 76: 177-88; Lorenzetti et al., 2001, J. Reconstr. Microsurg. 17: 163-7; Serletti et al., 2000, Semin. Surg. Oncol. 19: 264-71; Chang et al., 2000, Semin. Surg. Oncol. 19: 211-7; Chen et al., 1999, Hand Clin. 15: 541-54). These microvascular beds are considered expendable because they can be removed with no residual disability. Similar expendable vascular beds occur in animal models (Hoyt et al., 2001, Lab Anim. (NY) 30: 26-35; Zhang et al., 2001, J. Reconstr. Microsurg. 17: 211-21; Taylor et al., 1992, Plast. Reconstr. Surg. 89: 181-215). These microvascular beds are frequently composite tissues, such as bone and skin, muscle and skin, etc.

These microvascular beds can be removed, transferred to another location in the donor (or to an allogeneic recipient or host) and reintegrated into the systemic circulation using standard microsurgical techniques. Also known as “microvascular free flaps” or “microvascular free tissue,” these microvascular beds can support skin, bone, muscle or adipose tissue and are used clinically thousands of times each year in reconstructive surgery (Gurtner et al., 2000, Plast. Reconstr. Surg.106:672-82; quiz 683). They are employed to reconstruct ablative, congenital or traumatic defects in humans.

Replacement organs are critically needed for patients suffering from a multitude of diseases, including end-stage heart, lung and liver disease. At present, the only option for these patients is organ transplantation with the attendant problems of donor scarcity and life-long immunosuppression. The present invention provides methods for creating “neo-organs,” non-naturally occurring vascularized tissues that provide a function of a gland or organ, or that supplement the function of a gland or organ, from expendable, microvascular free flaps.

Citation or identification of any reference in Section 2 or in any other section of this application shall not be construed as an admission that such reference is available as prior art to the present invention.

### 3. SUMMARY OF THE INVENTION

The invention provides compositions and methods of ex-vivo modification of vertebrate tissue, e.g., by genetic transformation or by the introduction of cells, which is followed by implantation of the modified tissue into a patient in need thereof. Preferably, the tissue is microvascular flap (bed) tissue. A tissue explant is detached from the native circulation of a donor, transfected or modified ex vivo, and then attached (anastomosed) to a recipient, either the donor or another patient. Preferably, the tissue is manmalian tissue. More preferably, the vertebrate tissue is human tissue and the patient is a human patient. In embodiments in which genetic transformation modifies the tissue, modification is preferably via transfection with a nucleic acid encoding a product of interest. Such transfection is performed by contacting the selected tissue with a vector, preferably a viral vector, more preferably an adenoviral vector, that comprises the nucleic acid encoding the product of interest. The nucleic acid encoding the product of interest is driven by a regulatory element such as an inducible, constitutive or cell-specific promoter, preferably an inducible or constitutive promoter. After modification of the selected tissue via genetic transformation, the tissue is flushed to remove the vector not incorporated into the cells of the tissue. The selected tissue that is modified, by genetic transformation or by tissue engineering (e.g., introduction of cells), is then attached to the native circulation of the recipient using microvascular techniques. In one aspect, the invention provides methods of local delivery of a product (e.g., protein) of interest. In another aspect, the invention provides methods of systemic delivery of a product of interest. In yet another aspect, the invention provides methods of both local and systemic delivery of a product of interest.

The compositions and methods of the invention are particularly advantageous in that they avoid many of the problems associated with viral transfection of tissue in vivo. Using the methods of the invention, explanted microvascular flaps (or beds) can be modified ex vivo, via genetic transfection or introduction of cells, then reattached to the native circulation. In the case of genetically modified tissues, this enables high-level localized expression of the nucleic acid encoding a product of interest in cells of the explanted flap (or bed), with little or no collateral transfection occurring in other tissues. Current methods of human gene therapy, on the other hand, require systemic administration of a nucleic acid of interest (in vivo) or removal of isolated cells for modification (ex vivo) and subsequent re-infusion. Furthermore, the methods of the invention are reversible, in that the flap can be removed, in the event that the recipient displays an adverse reaction to the transplantation procedure or to the vector used to transfect the flap.

Furthermore, most donor organs are limited in availability. The flaps utilized by the methods of the invention, however, are obtained from expendable vascular beds. These flaps can be harvested from multiple donor sites with minimal or no finctional loss. The donor tissue may be autologous, avoiding the immunologic complexities of xenotransplantation. The ex vivo period permits modification of tissue, via genetic transformation or the introduction of cells, to provide therapeutic activity, and allows the flap to function as a delivery system for a therapeutic product, e.g., a protein. The compositions and methods of the invention therefore represent an advance over currently utilized techniques of delivery of nucleic acids and are ideal for targeted transfer of a nucleic acid of interest in patients undergoing microvascular flap transfers following ncologic surgery.

3.1. Definitions

As used herein, the term “bioreactor” refers to an ex vivo system for maintaining, ulturing, propagating, producing or expressing biological materials.

As used herein, the term “flap” refers to vascularized tissue for transplantation.

As used herein, the term “free flap” refers to vascularized tissue for transplantation hat is detached from the donor.

As used herein, the term “perfuse” or “perfusion” refers to the act of forcing a fluid to flow through the lumen of a hollow structure, e.g., forcing a fluid to flow from an artery or other blood vessel supplying a vascular bed of a tissue through the vascular bed of the tissue.

As used herein, the term “autologous” with respect to transplantation refers to a cell, tissue, organ, body part, etc. in which the donor and the recipient of the transplant are one and the same individual.

As used herein, the term “heterogenous” with respect to transplantation refers to a cell tissue, organ, body part, etc. in which the donor and the recipient of the transplant are different individuals.

As used herein, the term “pluripotent cell” refers to a primordial cell that may still have the capacity to differentiate into various specialized types of tissue elements.

As used herein, the term “primordial cell” refers to a cell from a group that constitutes the primordium of an organ or part of the embryo.

As used herein, the term “progenitor cell” refers to a cell that is committed to differentiate into a specific type of cell or to form a specific type of tissue.

As used herein, the term “stem cell” refers a precursor cell.

As used herein, the term “embryonic stem cell” refers to a stem cell that is derived from an embryo.

As used herein, the term “totipotent cell” refers to an undifferentiated cell capable of differentiating into any type of body cell.

As used herein, the term “neo-organ” refers to a non-naturally occurring vascularized tissue that provides a fulnction of a gland or organ, or that supplements the function of a gland or organ.

### 4. BRIEF DESCRIPTION OF THE FIGURES

FIGS. 1A-1E. Composite picture of surgical and flap infusion procedure. A. Schematic representation of the experimental process including flap harvest, ex vivo transfection and subsequent re-anastomosis to a different region for its therapeutic effect. B. Schematic of rat abdomen and groin with vascular supply. Area of flap elevation noted by—dashed line. C. Intra-operative photo and schematic of super-epigastric (SE) flap ex vivo. Note the arterial catheter and syringe is in place for viral infusion and a non-crushing clamp is on the vein. D. Intra-operative photo and schematic of SE flap after re-anastomosis to the native circulation. Note zoomed view in the schematic showing the arterial and venous anastomoses. Also note the femoral vessels entering from under the inguinal ligament. E. Intra-operative photo and schematic of SE flap sutured back in the groin after revascularization was completed. See Section 6 for details.

FIG. 2. Schematized drawing of a portion of microcirculation and of an expendable (micro)vascular bed (microvascular free flap). Microvascular beds are microcosms of the circulatory system. They contain all of the distinct, constituent cells that exist within the microcirculation. Grossly, they consist of large muscular arteries, leading to capacitance arterioles, endothelial lined capillaries, venules, veins as well as all of the phenotypically distinct cells within them. They contain all of these cell types in a functional and precisely ordered three-dimensional configuration. Since microvascular free flaps contain a single afferent artery and efferent vein, they can be easily reintegrated into the systemic circulation by standard vascular anastamoses.

FIG. 3. A schematized diagram of the general design of a bioreactor suitable for use in the methods of the invention. The bioreactor generally has four main mechanical components: pumps (labeled “1” and “1”), an oxygenator (labeled “2”), a tissue chamber (labeled “3”) and a control and monitoring module (labeled “4”). See Section 5.1.1 for details.

FIG. 4. A schematized diagram of one embodiment of the invention, in which an explanted (micro)vascular bed is maintained in a bioreactor. See Section 5.1.1 for details.

FIG. 5. Schematic diagram illustrating an exemplary intravascular route (A) and an exemplary extravascular route (B) used to deliver cells of interest to a microvascular bed. A. Bone marrow stem cells (e.g., hematopoietic or mesenchymal stem cells) are delivered to a microvascular bed via intravascular delivery. B. Mature differentiated cells (e.g., hepatocytes) are delivered to a microvascular bed via extravascular delivery. In both examples, cell fate and viability is determined after delivery and ex vivo maintenance of the cells in the microvascular bed.

FIGS. 6A-6D. A and B. Photomicrographs of histologic sections stained with X-gal. Two groups received AdCMVLacZ at a concentration of either 1×10⁶ PFU/ml (i.e., plaque forming units/ml or viral particles/ml) or 4×10¹¹ PFU/ml. In panel A, the section is from an animal that received AdCMVLacZ at a concentration of 1×10⁶ PFU/ml. In panel B, the section is from an animal that received AdCMVLacZ at a concentration of 4×10¹¹ PFU/ml. Note, in panel B, the increased (darker) staining (indicating increased gene expression and β-gal activity) at the higher concentration. The staining is noted in all tissue levels of the flap. C. Gene expression after viral infusion at different concentrations. As noted, two groups received AdCMVLacZ at a concentration of either 1×10⁶ PFU/ml or 4×10¹¹ PFU/ml. Note the dose-response difference in gene expression at all cell levels. Also note the high levels of transfection, in all tissue levels, obtained with this transfection protocol. D. Gene activity levels from time of injection at concentration of 4×10¹¹ PFU/ml. Note highest level of gene expression at 5 days with persistence through 14 days and subsequent drop-off of expression. See Section 6 for details.

FIGS. 7A-7D. A. Second infusion protocol in which the viral concentration was 2.5×10⁹ PFU/ml and the efferent vein was left clamped. Photoricrograph of flap sample at 40× magnification. Note stained cells representing β-gal activity. B. Photomicrographs of all harvested tissue types at comparative magnification. First section is flap tissue at 20× magnification. Again note staining in the tissue shown. Second section (20× magnification) is contralateral skin in the same animal. Note no staining of the tissue. Third section (20× magnification) is a liver specimen from the same animal. Note no staining of the tissue. The fourth section (20× magnification) is a spleen specimen in the same animal. Again, note no blue staining of the tissue. C. PCR of flap and non-flap tissues. Note the signal obtained within the viral, control lane and the flap itself but no signal in the non-flap tissue. This is representative of the localization of gene activity primarily to the flap. D. β-Gal ELISA of flap and non-flap tissues in the same experimental group using viral concentration of 2.5×10⁹ PFU/ml compared with an animal that had a single acute bolus injection of the same total amount of the virus (in terms of total PFU) given systemically through the tail vein. Note the higher localization of β-gal protein within the flap tissue compared to the background levels of the other non-flap tissues in the experiment. Also note that although β-gal protein levels were found in all tissues of the systemic injection group there was no level as high as the activity found in the transfected flap itself. See Section 6 for details.

### 5. DETAILED DESCRIPTION OF THE INVENTION.

In one aspect, the invention provides methods of genetically transforming selected vertebrate tissue ex vivo, then reimplanting the tissue into a patient or recipient in need of the transformed tissue. The vertebrate may be a mammal, bird, reptile, amphibian, etc. In certain embodiments, the vertebrate is a mammalian species such as mouse, rat, rabbit, guinea pig, dog, cat, pig, sheep, cow, horse, monkey, etc., and in a preferred embodiment, it is a human.

Preferably, the tissue is microvascular bed (or flap) tissue, and the tissue explant is detached from the native circulation, transfected ex vivo, and then reattached (reanastomosed). In a preferred embodiment, the tissue is mammalian tissue. In a more preferred embodiment, the tissue is human tissue and the patient is a human patient. Transfection with a nucleic acid encoding a product of interest is performed by contacting the selected tissue with a vector, preferably a viral vector, more preferably an adenoviral vector, that comprises the nucleic acid encoding the product of interest. The expression of the nucleic acid encoding the product of interest is driven by regulatory element such as an inducible, constitutive or cell-specific promoter, preferably an inducible or constitutive promoter. In a preferred embodiment, the promoter. is a CMV promoter. After genetic transformation of the selected tissue, the tissue is flushed to remove any vector not taken up by the cells of the tissue, and then reattached to the native circulation using microvascular techniques.

The methods of the invention are particularly advantageous in that they avoid many of the problems associated with transfection of tissue in vivo. Using the methods of the invention, the inventor has made the surprising discovery that explanted microvascular beds (or flaps) can be transfected ex vivo with a viral vector comprising a nucleic acid encoding a product of interest and reattached to the native circulation using microvascular techniques, enabling high regional expression of the nucleic acid encoding a product of interest, which can be observed in the cells of the explanted flap (or bed). High-level transgene expression can be precisely localized to the explanted flap with no collateral transfection occurring in other tissues. Currently, human gene therapy requires systemic administration of a nucleic acid of interest (in vivo) or removal of isolated cells for modification (ex vivo) and subsequent re-infusion. The compositions and methods of the invention represent an advance over currently utilized techniques of nucleic acid delivery and are ideal for targeted transfer of a nucleic acid of interest in patients undergoing microvascular flap transfers following surgery, in particular, oncologic surgery.

In another aspect, the reattaching step is accomplished using microvascular surgical techniques.

In another aspect, the invention provides methods of chronic or continuous systemic delivery of a bioactive molecule to a patient.

In another aspect, the invention provides methods for producing a “neo-organ,” i.e., a non-naturally occurring vascularized tissue that provides a function of a gland or organ, or that supplements the function of a gland or organ, that delivers locally or systemically a product of interest to a patient in need thereof.

In another aspect, a microvascular free flap is genetically modified ex vivo with a nucleic acid encoding a therapeutic molecule of interest, and the flap functions as a neo-organ that delivers the molecule of interest following re-implantation and re-anastomosis.

In another aspect, the invention provides methods of genetically transforming selected vertebrate tissue with a nucleic acid encoding a product of interest comprising detaching the tissue from the native circulation using microvascular surgical techniques; transfecting the tissue ex vivo by perfusing the tissue with a suspension comprising an adenoviral vector wherein-the adenoviral vector comprises the nucleic acid encoding the product of interest under the control of a CMV promoter; and reattaching the tissue to the native circulation using microvascular surgical techniques. In certain embodiments, the vertebrate tissue is mammalian tissue. In a preferred embodiment, the tissue is human tissue. In another preferred embodiment, the concentration of the suspension of adenoviral vector is 2.5×10⁹ to 4×10¹¹ PFU/ml.

In yet another aspect, the microvascular free flap is modified ex vivo by the introduction of cell(s) of interest. For example, a population of cells can be “seeded” or established in the microvascular free flap by introducing, e.g., autologous cells, heterogenous cells, pluripotent cells, primordial cells, stem cells, embryonic stem cells, totipotent cells, differentiated cells, etc. In certain embodiments, the cells are introduced into microvascular free flap that is maintained in a maintenance system such as a bioreactor, and the cells are introduced into the microvascular free flap by e.g., perfusion. In certain embodiments, the microvascular free flap with associated cells is returned to a patient or recipient in need thereof, e.g., by transplantation into the recipient using standard microvascular techniques. In certain embodiments, the introduced cells are derived from stem cells that have differentiated ex vivo.

For clarity of disclosure, and not by way of limitation, the detailed description of the invention is divided into the subsections set forth below.

5.1. Methods of Tissue Harvest

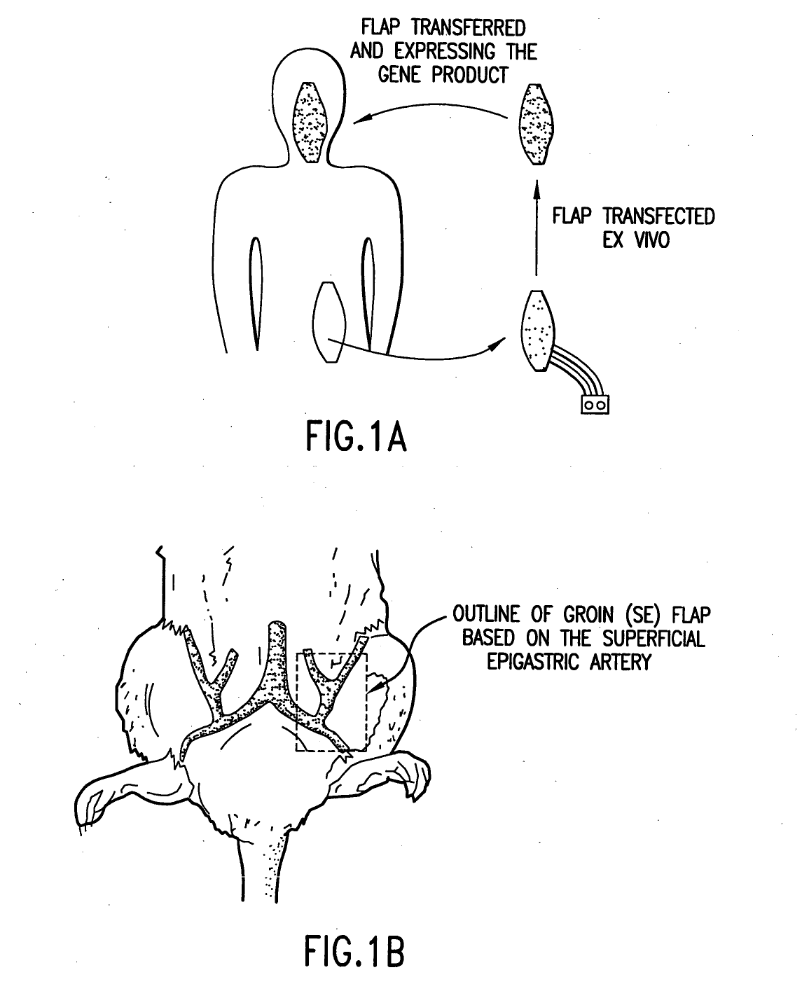

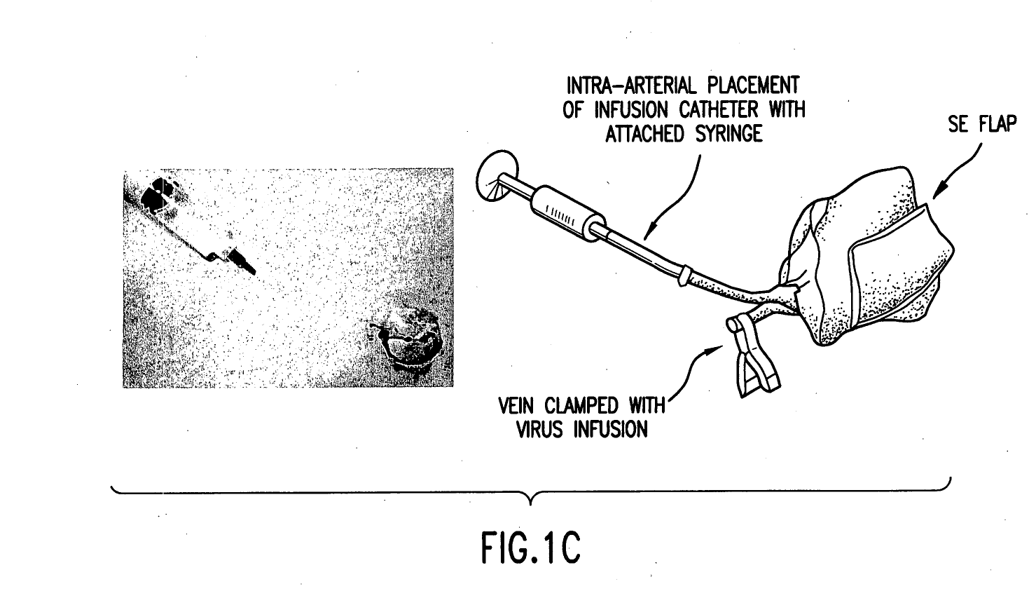

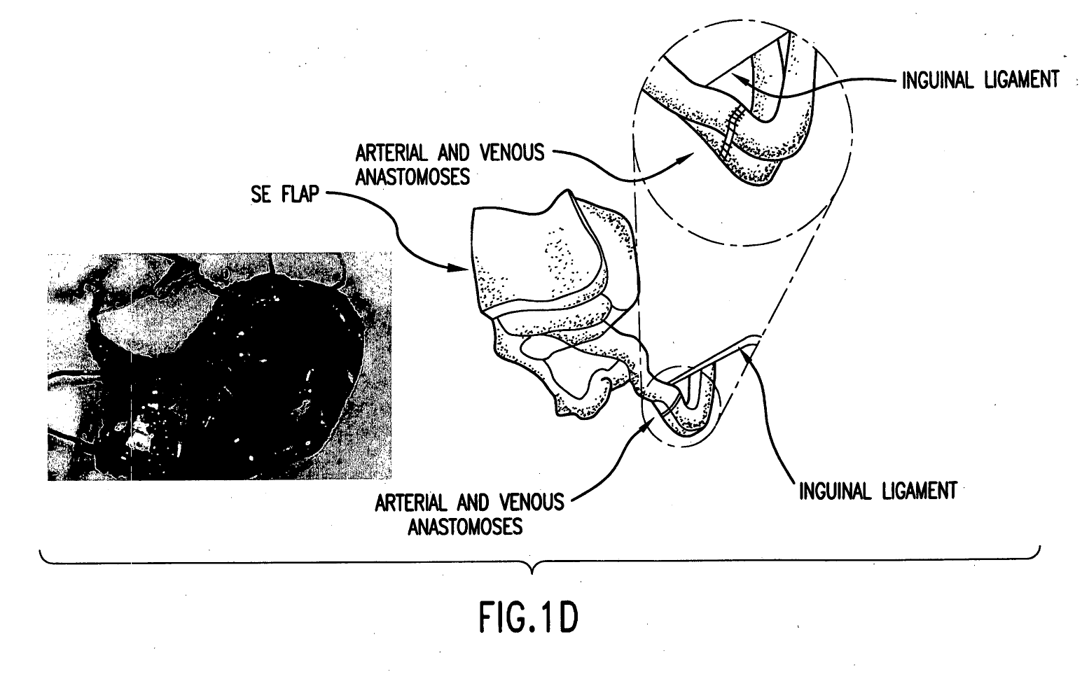

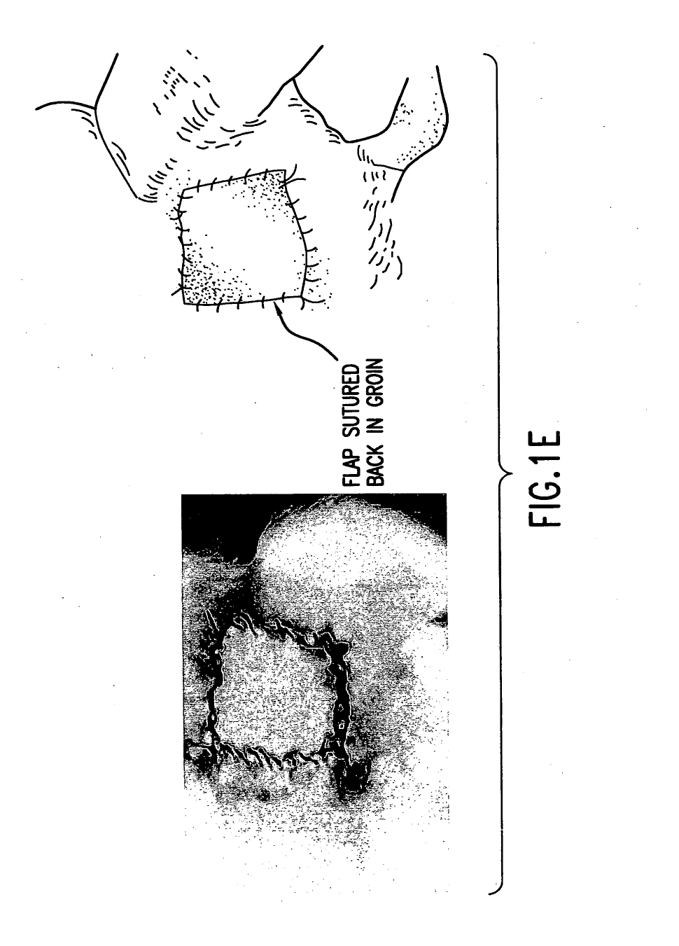

According to the methods of the invention, a tissue of interest is harvested as an explant for modification and subsequent reattachment or reanastomosis. Preferably, the tissue of interest is a microvascular bed or microvascular “free flap” (MVFF). A microvascular bed or free flap is an intact microcirculatory network or bed. Microvascular free flap transfer is the auto-transplantation of composite tissues (known as a free flap) from one anatomic region to another (Blackwell et al., 1997, Head Neck 19: 620-28). Clinically, it is routinely performed to reconstruct defects following tumor extirpation such as in a mastectomy. In performing microvascular free flap transfer, an intact microcirculatory network or bed is detached. According to the methods of the invention, this vascular network is detached from the intact organism for a finite period of time (ex vivo), and undergoes modification, e.g., by genetic modification or tissue engineering, and in a preferred embodiment, by transfection (FIG. 1A). This provides an opportunity to manipulate and modify this tissue without risk of systemic toxicity.

Microvascular free flap transfer generally entails the division and subsequent re-anastomosis of the dominant artery and vein in the composite tissue (flap), allowing the transplanted tissue to survive. As such, microvascular free tissue transfer represents the manipulation and transfer of an intact microcirculatory network or bed. This network can supply a variety of tissues because of its functioning microcirculatory network. This vascular network may be detached from the intact organism and maintained ex vivo, permitting its manipulation or modification without danger of systemic toxicity.

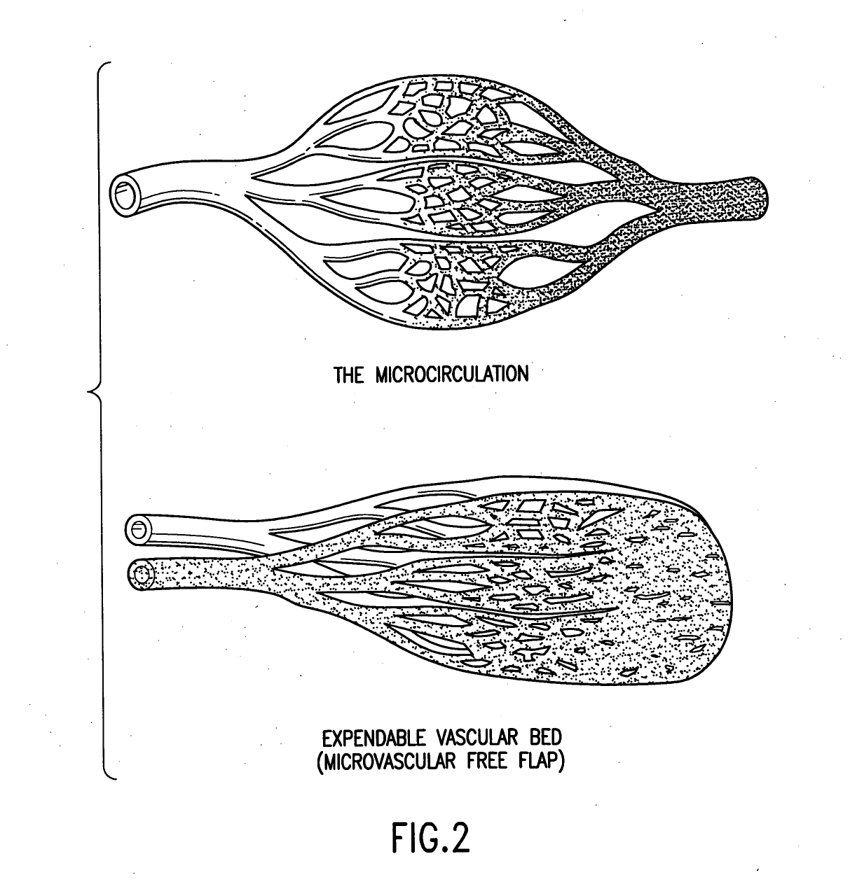

These expendable microvascular beds are illustrated in FIG. 2. When in their normal, native state, they contain all of the distinct, constituent cells that exist within the microcirculation (Krapohl et al., 1998, Plast. Reconstr. Surg.102: 2388-94; Taylor et al., 1987, Br. J. Plast. Surg. 40: 113-41). Grossly, they consist of large muscular arteries, leading to capacitance arterioles, endothelial lined capillaries, venules, veins and all of the phenotypically distinct cells within them (Siemionow et al., 1998, Ann. Plast. Surg. 41: 275-82, Carroll et al, 2002, Head Neck. 22: 700-13). Importantly, in the native state, they contain all of these cell types in a functional and precisely ordered three-dimensional configuration. In a sense, they have already been “patterned”. These expendable microvascular beds provide an ideal, living substrate on which to fabricate a “neo-organ,” i.e., a non-naturally occurring vascularized tissue that provides a function of a gland or organ, or that supplements the function of a gland or organ. Since microvascular free flaps contain a single afferent artery and efferent vein they can be easily reintegrated into the systemic circulation by standard vascular anastamoses.

According to the methods of the invention, a selected tissue may be excised (“harvested”) by conventional surgical methods known in the art (see, e.g., Petry et al., 1984, Plast. Reconstr. Surg. 74: 410-13; Blackwell et al., 1997, Head Neck 19, 620-28). The surgical procedure results in the removal of skin and subcutaneous tissue associated with blood vessels in a select region of the body. For example, the flap can be a superepigastric (“SE” or lower abdomen/groin) flap and the associated blood vessels can be SE blood vessels of the lower abdomen and groin.

In another aspect of the invention, a composite tissue flap, i.e., a flap composed of bone and skin, muscle and skin, adipose tissue and skin, fascia and muscle, or other such combination known to normally be present in the vertebrate body, is used because it has a greater tolerance for ischemia, allowing for more extensive genetic manipulation prior to re-anastomosis.

Once the flap is excised, the proximal blood vessels that are associated with the flap are clamped while the flap is ex vivo. Any conventional technique known in the art can be used to clamp the blood vessels.

The selected tissue is maintained ex vivo by methods for maintaining explants well-known in the art. The tissue is preferably perfused, e.g., the tissue can be wrapped in gauze, a catheter can be placed in a blood vessel associated with the tissue and secured with a suture, and the tissue perfused or infused with physiological saline. In one embodiment, the perfusion is conducted at a cold temperature (for cold ischemia). In other embodiments, perfusion is conducted at room temperature or body temperature. Preferably, the tissue is perfused ex vivo through a catheter at a constant perfusion pressure to flush out blood from the flap vessels. Preferably, the infusions are given at physiologic pressures (80-200mm Hg), since high pressures cause excessive tissue damage, leading to necrosis of all or part of the flap. In one embodiment, a continuous microperfusion system, such as the one described by Milas et al. (1997, Clinical Cancer Research. 3(12-1): 2197-2203) is used.

In other embodiments, an explanted flap can be explanted and maintained for a prolonged period of time by using an immunodeficient host as a recipient.

5.1.1. Bioreactor

In certain embodiments, the harvested issue, e.g., a microvascular free flap, is explanted and maintained ex vivo in a bioreactor.

In one aspect of the invention, a microvascular free flap is removed from a donor and perfused ex vivo by incorporating the tissue within the bioreactor via its afferent artery and efferent vein. The microvascular network which exists within the closed circuit defined by these two vessels can be manipulated to eventually support the growth of new organs and tissues.

Technology well known in the art can be used to perfuse a microvascular free flap in a bioreactor. The flap may be maintained in the bioreactor for a short period of time, e.g., one hour to one day, or can be maintained for a longer period of time, e.g, one week, one month, six months, one year or indefinitely.

A bioreactor suitable for use in the methods of the invention provides perfusion of the tissue, delivery of nutrients and oxygen, and removes wastes within the confines of a sterile environment. Similar design and engineering concepts have been successfully used in the construction of bioreactors, hepatic assist devices and organ maintenance or preservation systems and are well known in the art (Wolfe et al., 2002, Biotechnol. Bioeng. 77: 83-90; Tilles et al., 2001, Biotechnol. Bioeng. 73: 379-89; De Bartolo et al., 2002, Biotechnol. Prog. 16: 102-8; Gerlach et al., 1994, Transplantation 58: 984-8; Allen et al., 2001, Hepatology 34: 447-55; Tzanakakis et al., 2000, Annu. Rev. Biomed. Eng. 2: 607-32).

A bioreactor suitable for use in the methods of the invention has the following properties, which are well known in the art as general properties of a bioreactor:

1. An ability to transport oxygen and nutrients to the cells/tissue

2. A means to remove metabolic wastes and toxins from the tissue bed

3. A means to add and remove nutrients/growth factors

5. A means to add cells (e.g., stem cells) to the system

6. A means to monitor cellular viability

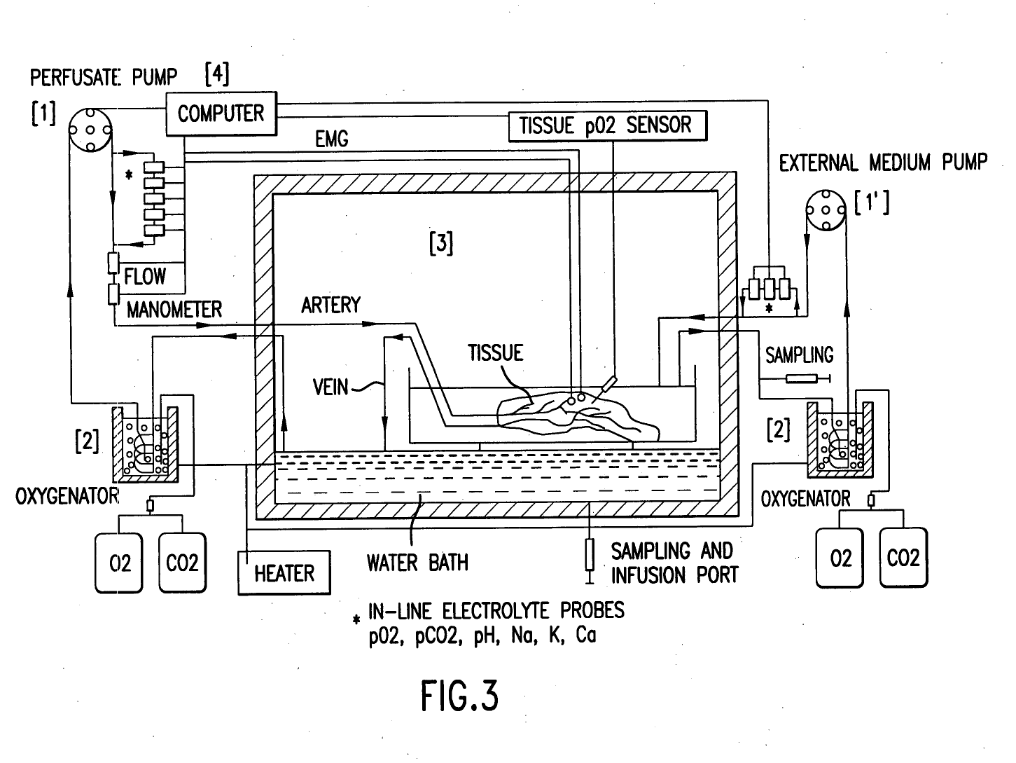

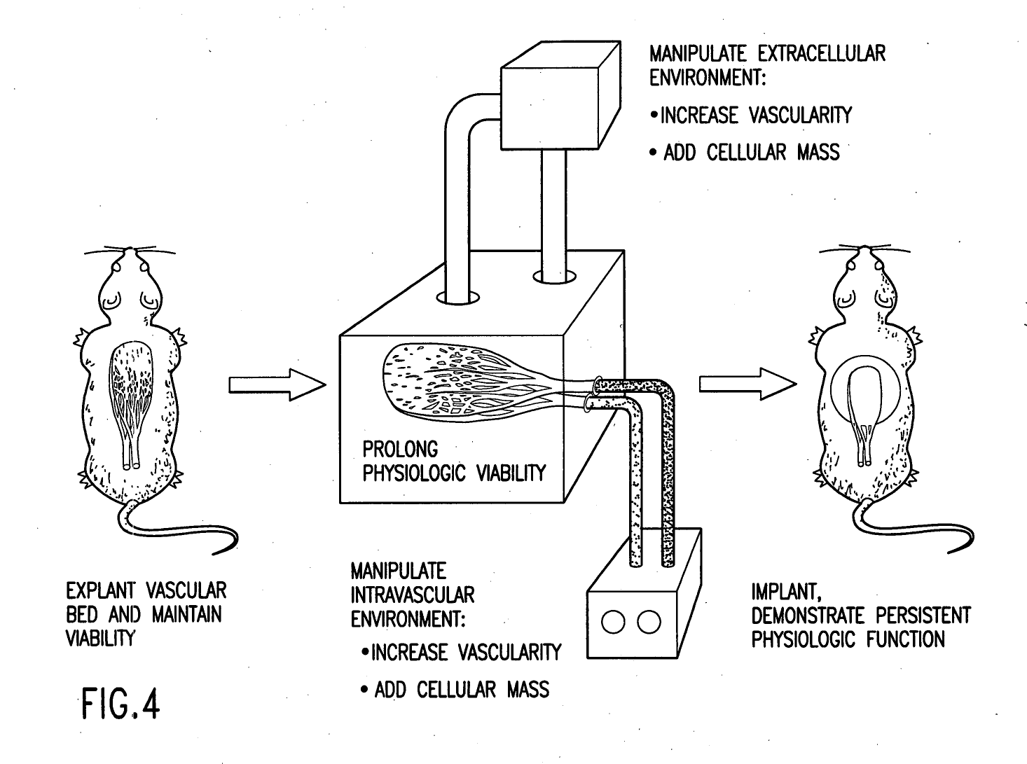

Schematically, the main components of a bioreactor suitable for use in the methods of the invention can be divided into three areas (see FIGS. 3 and 4):

1. Biologic—i e., a tissue bed, e.g., a microvascular free flap, that is to be utilized and kept viable.

2. Mechanical—the hardware (engineering) aspects of the bioreactor.

3. Metabolic—the media and perfusion solutions, growth factors, nutrients that are delivered by the mechanical components to nourish the biologic part.

Tissue viability and any damage to, or deterioration of, tissue can be monitored by measuring the effluent fluid. For tissue comprising muscle tissue, e.g., quadriceps femoris muscle, the effluent fluid can be monitored for lactate levels as well as creatine phosphokinase (CPK) levels. Endpoints for maintenance in the bioreactor can be determined by measuring these two indicators of viability.

In certain embodiments, a flap is transfected while being maintained in a bioreactor. Following transfection, flaps can be flushed with perfusion medium (e.g., University of Wisconsin (UW) medium). The variables that can be examined include temperature of the flush (4° C., 20° C., 37° C.) and volume of flush (5-250 cc) (Franken et al., 1999, Microsurgery 19(5): 214-22). Positive controls can consist of animals in which the vector of interest is injected e.g., via intravenous or intraperitoneal injection (i.e., in vivo) and negative controls can consist of animals that receive no vector in the initial perfusion solution. Control animals can be sacrificed and organs can be harvested at 7 days, near the peak of adenoviral expression. Specimens from flaps transfected while being maintained in the bioreactor and from controls can be examined for presence of a gene of interest or expression thereof, e.g., with X-gal staining, antibody staining, PCR, etc.

PCR is preferably employed on negative specimens to confirm the absence of any viral DNA sequences in tissues other than the flap and recipient bed. Histologic analysis using standard art-known methods can be employed to define any toxicity produced by the flushing protocol. Preferably, the gene product is present in the treated flap and the local recipient bed, but not in any of the other tissues examined from the recipient.

A bioreactor suitable for use in the methods of the invention generally has four main mechanical components (FIG. 3).

Pumps: (Labeled “1” and “1” in FIG. 3): Any pump that is known in the art as suitable for use in a bioreactor may be used. In one embodiment, a variable speed roller pump (“1”) can be used as described by Stangl et al. (2000, Eur. Surg. Res. 32: 100-6) to deliver the tissue perfusion solution into the afferent artery of the microvascular bed. The speed of the pump is adjusted by a feedback system utilizing a manometer and special computer software to maintain the perfusion pressure within a tight range. A second pump (“1”) is used to circulate the solution around the tissue. Pressures lower than 15-30 mm Hg cause decreased oxygen supply (Gohra et al., 1989, Ann. Thorac. Surg. 48: 96-103; Toledo-Pereyra et al., 1979, Ann. Thorac. Surg. 27: 24-31), whereas excessively high pressures (greater than 200 mm Hg) can damage the capillary bed and cause edema (Kioka et al., 1986, J. Heart Transplant. 5: 437-43). At a constant pressure, the flow rate is monitored to assure adequate perfusion of the tissue. The pressure used may be determined using methods commonly known in the art (see below). pO₂ is also measured and recorded within the block of tissue, using routine methods, as a further gauge of adequate perfusion.

Oxygenator (labeled “2” in FIG. 3): The membrane lung can consist of any material known in the art as suitable for use in a bioreactor, e.g., silicon tubing in a glass container, according to the methods described in Hamilton et al. (1974, J. Lipid Res. 15: 182-6). The necessary gas mixture (a calibrated mixture of oxygen and carbon dioxide) is directed into the glass container to achieve the adequate level of oxygenation. Oxygen partial pressure is monitored distal to the oxygenator. Contribution of the different gases, as well as the flow rate of the mixture is adjusted to maintain adequate gas partial pressures. The oxygenator is kept in a water bath maintained at the desired temperature.

Tissue Chamber (labeled “3” in FIG. 3): Any tissue chamber that is known in the art as suitable for use in a bioreactor may be used. In a specific embodiment, the tissue chamber consists of two fluid containers one within the other with separate perfusion systems. The smaller container houses the tissue and the external bathing solution. The larger container is double-walled and holds the first container and also a reservoir for the overflow of the perfusion solution draining from the vein. The external container is kept at the desired temperature via a water bath. The containers are connected to the rest of the system via silicon tubing. There may be additional ports for further instrumentation (e.g., tissue oxygen probe (Oxy-Lite, Oxford Optronix), temperature probe). In certain embodiments, the media bathing the tissue and the solutions delivered intravascularly may be independent of each other.

Control and monitoring module (labeled “4” in FIG. 3): Any control and monitoring module that is known in the art as suitable for use in a bioreactor may be used. In a specific embodiment, a computer system with amplifiers, signal processing hardware and software is used to monitor and record pressure, flow rate, temperature, pO₂, pCO₂, Na⁺, K⁺, pH with in-line probes. Samples are obtained at regular intervals to measure glucose, CPK and LDH levels. Because of the modular design, the system has the flexibility to measure and record additional parameters. The electrical activity and response of the muscle is also monitored as an indicator of the functional status of the muscle tissue.

The metabolic components of a bioreactor that is suitable for use in the methods of the invention generally are as follows:

Perfusion solution: While whole blood may be the “perfect” perfusion fluid in vivo, it has many problems limiting its use ex vivo. Particular problems associated with the use of whole blood include, but are not limited to, red blood cell destruction and lysis in a mechanical system, the need for constant replacement, the need for anticoagulation and the presence of other cellular mediators of inflammation which may be deleterious to the survival of a microvascular bed.

In certain embodiments of the invention, a modified Krebs's solution (used in organ maintenance or preservation studies) can be used. The solution may be supplemented with plasma, serum, media with serum and media with.reduced amounts or even serum-free media containing cocktails of one or more growth factors, etc. The exact composition of the media/perfusion solution may determined experimentally using methods well known in the art.

Oxygen: Although most cells are grown in vitro at atmospheric oxygen concentrations (20.9%), most cells in the body are exposed to oxygen concentrations of only 3-7%. Indeed, it is commonly known in the art that in order for a vasculature to properly develop in the embryo, that oxygen concentrations must be less than 10% (Chen et al., 1999, Teratology 60: 215-25). Therefore, in certain embodiments of the invention, oxygen concentrations are varied, using methods well known in the art, to determine which O₂ tension results in optimal tissue viability.

Albumin: In certain embodiments of the invention, albumin is included in the perfusion solution. Albumin, when included in perfusion solutions, contributes to oncotic pressure, particularly in serum-free solutions. It reduces edema formation during perfusion (Wicomb et al., 1982, Transplantation 34: 246-50; Segel et al., 1992, Am. J. Physiol. 263: H730-9). Osmotic pressures in the range of 300 to 370 mOsm/L have been reported to be beneficial in organ perfusion systems (Corno et al., 1987, J. Thorac. Cardiovasc. Surg. 93: 163-72; Schaff et al., 1981, Surgery 89: 141-50). In certain embodiments of the invention, a concentration >5 gm/L of albumin may be used in the perfusion solution, e.g., by using bovine albumin supplementation, to achieve a desired osmolarity and oncotic pressure.

Electrolytes: Near physiologic concentrations of the major electrolytes are preferably chosen as a starting point for the composition of the perfusion fluid. The concentration of potassium in Krebs's solution, for example, is 4 mmol/l and is maintained in the range of 3 to 4 mmol/l. Normocalcemic solutions lead to accumulation of calcium in the cells during ischemia and reperfusion (Kronon et al., 1997, J. Thorac. Cardiovasc. Surg. 114: 1010-9). Calcium-free perfusion solutions lead to an increase of calcium uptake during reperfusion. This can result in irreversible cell damage (Kirkels et al., 1989, Circ. Res. 64: 1158-64). Hypocalcemic solutions can improve the results during cardiac perfusion. The starting concentration of calcium in Krebs's solution is 2.5 mmol and in certain embodiments, calcium is maintained in the range of 1.5 to 2.0 mmol/l. Magnesium is effective in preventing some of the negative effects on intracellular calcium accumulation (Kronon et al., 1997, J. Thorac. Cardiovasc. Surg. 114: 1010-9). The starting concentration of magnesium is 1 mmol/l in Krebs's solution and is preferably maintained at the same level. The concentrations of all these electrolytes are similar to those found in commonly used tissue culture media.

Energy substrates and nutritional supplements: In certain embodiments, glucose is included in the perfusion solution. Glucose has a cardioprotective-effect during perfusion maintenance or preservation, and insulin enhances the effect of glucose (de Wit et al., 1988, J. Thorac. Cardiovasc. Surg. 95: 310-20; Steinberg et a., 1991, Ann. Thorac. Surg. 51: 620-9). Insulin and glucose can have deleterious effects, however, if certain conditions are not met, e.g., an adequate oxygen supply and removal of metabolic products (Steigen et al., 1993, Acta Physiol. Scand. 149: 143-51; Zhu et al, 1994, J. Heart Lung Transplant. 13: 882-90). In a specific embodiment, an initial concentration of 200 mg/dl dextrose in Krebs's solution is maintained. Insulin concentrations may be titrated using methods commonly known in the art to maintain near normal oxygen consumption.

Temperature: Temperatures close to 5° C. are commonly used in the art for tissue maintenance (Proctor et al., 1968, Br. Med. J. 4: 296-8). Lower temperatures decrease the tissue energy requirements, but metabolic activity can still occur at low temperatures. Hypothermic conditions, however, can adversely affect physiologic functions of the stored organs and tissues. Warm perfusion can enhance the tissue protective effects of metabolic substrates such as glucose, insulin, and aspartate, because their utilization is possible during aerobic metabolism (Kober et al., 1996, Transplant Proc. 28: 160-2). Therefore, in certain embodiments of the invention, tissue is preferably maintained at normal temperatures (37° C.).

Acid-Base status: Sterile sodium bicarbonate solution or other art-known solution may be used to titrate the pH close to pH 7.47 (Carter et al., 1980, Transplantation 30: 409-10).

Formulation of perfusion solution: The maintenance or culture medium that is best suited for maintenance of tissue viability within the bioreactor may be determined using any method commonly known in the art. Any maintenance solution or culture medium that is suitable for use in a bioreactor or for perfusion of tissue can be used, including, but not limited to, modified Krebs's solution with and without bovine serum, bovine plasma, Modified Eagle's Medium with serum (Gibco BRL), or endothelial specific growth medium (EGM-2-MV, Clortetics Corporation). To optimize perfusion conditions, tissue can be perfused by one of these perfusion solutions and examined at 30 minutes, 1 hour, 2 and 3 hours via biopsies for histological studies. At the same time points, samples of the perfusion solution can be obtained for measurement of CPK, glucose, and lactate levels. If a microvascular free flap maintained in the bioreactor contains muscle, then the electrical response of the muscle can be recorded prior to biopsy.

Numerous categories of perfusion solutions known in the art may be employed. For example, one category may be based on the well-known Krebs's solution. A second category may be based on common cell culture media. Oxygen carrying capacity of such perfusion solutions may not meet the metabolic demands of the tissue. Therefore, the system can be monitored via measurements of lactate and oxygen consumption to recognize this problem. If such a situation arises, solutions containing perfluorocarbon emulsions with significant higher oxygen carrying capacity may be used. Heme-based solutions may also be used, if the perfluorocarbon-based solutions are inadequate for long-term tissue maintenance (Benesch et al., 1984, Proc. Natl. Acad. Sci. USA. 81: 2941-3).

Oxygenation and perfusion pressure: suitable pressure (physiological (i.e., 100-120 mm Hg) versus sub-physiological) and oxygen concentrations (1-20%) that enhance tissue survival can be determined using any method commonly known in the art. In a specific embodiment, the same endpoints of CPK, glucose and lactate. in the effluent are measured.

Prolonged tissue maintenance: Using a perfusion solution formulated as described above, long-term tissue viability studies (ranging from 1-7 days) are performed. In each experiment, four time points are chosen. The perfusion solution is analyzed for CPK, glucose and lactate levels. Tissue biopsy studies and electrical stimulations are performed as described above. With increasing length of maintenance, the time intervals for measurements may be likewise extended.

Risk of infection: With increasing maintenance time, the chance of infection also increases. The entire process of tissue harvest and placement into the tissue chamber of the bioreactor is preferably performed with strict sterile techniques. Antibiotics, such as penicillin G and streptomycin, are preferably used prophylactically. The bioreactor system is preferably sterilized after each use (autoclave or ethylene oxide sterilization depending on the material).

Metabolic wastes: During short periods of perfusion maintenance, metabolic wastes do not accumulate in sufficient amounts to require active elimination. During longer perfusion times, metabolic end products may need to be removed. The use of readily available perfusion solutions permits the gradual exchange of the perfusion solution with increasing perfusion time. This keeps the concentration of metabolic byproducts to a minimum. Ultra filtration of the perfusion solution may also be used, especially for blood-based perfusion solutions. In certain embodiments, a dialysis module may be added into the perfusion system to clear the metabolic wastes.

Reperfusion injury: During the harvest of the tissue and maintenance, there may be periods of tissue ischemia with reperfusion injury. The ischemic period is minimized by streamlining the tissue harvest and transfer protocols. If reperfusion injury occurs, well known pharmacologic agents may be used to minimize it (Land et al., 1994, Transplantation 57: 211-7;Nicolini etal., 1991, Am. Heart J.122: 1245-51).

In certain embodiments, SigmaStat software (Jandel Scientific) is used to perform statistical tests to analyze experimental conditions in the bioreactor. Student's t-test as well as ANOVA are utilized. At least three different measurements at independent time points are preferably taken of all variables.

Optimization of bioreactor conditions: The following algorithm may be carried out to optimize maintenance conditions in the bioreactor.

1) Perfuse a tissue bed or microvascular free flap and attain homeostasis.

2) Change one specific variable (e.g., pressure).

3) Sample effluent from the efferent vein.

4) Assay effluent for pH, glucose, CPK and lactate levels.

5) Examine select tissue samples histologically.

6) Adjust experimental variable on the basis of the results.

By using the above simple algorithm, manipulations can be identified that enhance tissue viability, that adversely affect tissue survival, or that have no effect.

In certain cases, cells that are delivered to a rnicrovascular free flap maintained in a bioreactor using the methods of the invention may adhere, clump, or clog the intravascular space. A bioreactor system suitable for use in the methods of the invention will, in certain embodiments, permit the continuous perfusion of the microvascular bed, and therefore will able to perfuse a diluted suspension of cells through the circuitry and still achieve high rates of incorporation within the tissue.

Tissue can survive ex vivo for a short time (i.e.,hours) with no significant effect on vascular patency and cellular function following re-implantation. Longer periods of ex vivo maintenance may, in some instances cause microvascular flap-failure (i.e., thrombosis, endothelial damage, and/or edema). These conditions are assessed by the clinical judgment of the ordinarily skilled practitioner, as well as by, e.g., histological evaluation with standard histological sections taken from both proximal, middle, and distal microvascular bed and surrounding normal tissue.

5.2. Methods of Tissue Modification

According to the methods of the invention, the harvested tissue explant undergoes modification ex vivo. In one embodiment, it undergoes genetic transformation, preferably by transfection with a viral vector. Transfection may be accomplished by methods well known in the art. A variety of transformation or transfection techniques are currently available and used to transfer DNA in vitro into cells, including calcium phosphate-DNA precipitation, DEAE-dextran transfection, electroporation, liposome-mediated DNA transfer or transduction with recombinant viral vectors, lipofectin, infection, microinjection, cell fusion, lysosome fusion, synthetic cationic lipids, and use of a gene gun or a DNA vector transporter. For various techniques for fransformation or transfection of mammalian cells, see Keown et al., 1990, Methods Enzymol. 185: 527-37; Sambrook et al., 2001, Molecular Cloning, A Laboratory Manual, Third Edition, Cold Spring Harbor Laboratory Press, N.Y. Such ex vivo treatment protocols can be used to transfer DNA into a variety of different cell types including epithelial cells (U.S. Pat. No. 4,868,116; Morgan and Mulligan WO87/00201; Morgan et al., 1987, Science 237:1476-1479; Morgan and Mulligan, U.S. Pat. No. 4,980,286), endothelial cells (WO89/05345), hepatocytes (WO89/07136; Wolff et al., 1987, Proc. Natl. Acad. Sci. USA 84:3344-3348; Ledley et al., 1987 Proc. Natl. Acad. Sci. 84:5335-5339; Wilson and Mulligan, WO89/07136; Wilson et al., 1990, Proc. Natl. Acad. Sci. 87:8437-8441) fibroblasts (Palmer et al., 1987, Proc. Natl. Acad. Sci. USA 84:1055-1059; Anson et al., 1987, Mol. Biol. Med. 4:11-20; Rosenberg et al., 1988, Science 242:1575-1578; Naughton & Naughton, U.S. Pat. No. 4,963,489), lymphocytes (Anderson et al., U.S. Pat. No. 5,399,346; Blaese, R. M. et al., 1995, Science 270:475-480) and hematopoietic stem cells (Lim, B. et al., 1989, Proc. Natl. Acad. Sci. USA 86:8892-8896; Anderson et al., U.S. Pat. No. 5,399,346).

In a preferred embodiment, the selected tissue, preferably a free flap, is transfected by perfusion with a suspension of a viral vector, wherein the vector comprises a recombinant nucleic acid encoding a product of interest. Preferably, the titer of the suspension is approximately 2.5×10⁹ to 4×10¹¹ PFU/ml in order to bring about efficient transformation of the-tissue. For an average-sized (e.g., 75 kg) human, a total of 10-50 ml, preferably approximately 30 ml of vector suspension is perfused into the selected tissue.

In certain embodiments, the free flap is perfused with a high titer of viral vector (>10¹⁰ total PFU).

In another embodiment, the vector is introduced into the microcirculation of the flap for at least one hour. Subsequently, vector that has not been taken up by the cells of the flap is preferably flushed out of the flap vasculature by perfusion.

Variables that can be optimized according to methods well known in the art include concentration of the vector infused, e.g., adenovirus (10⁸-10¹² PFU); the duration of perfusion (e.g., 5 min to 2 hours) and the temperature of perfusion solution (preferably in the range of 4° C.-37° C.).

In one embodiment, the tissue explant is perfused through an arterial catheter, and the vein associated with the tissue is left open to allow outflow of the suspension or solution. In another embodiment, the vein is clamped to occlude outflow and the tissue is perfused through the arterial catheter until the vein is distended. In a preferred embodiment, the vein is maintained in a distended state by intermittently adding aliquots of perfusion suspension comprising the vector. The suspension of vector is preferably allowed to incubate or dwell in the flap between infusions and for up to one hour after completion of the infusion.

After perfusion (or infusion) with the vector suspension and incubation (if any) after perfusion, the venous clamp (if any) is removed and the tissue flap is flushed with physiological saline and surfaces rinsed to remove the perfusion suspension containing the vector. Preferably, the tissue preparation is washed or flushed thoroughly so that no vector contacts other (non-targeted) tissues.

In another embodiment, the tissue explant is modified ex vivo by tissue engineering. General methods of tissue engineering are well known in the art (Vacanti et al., 1999, Lancet 354 Suppl. 1: S132-34; Vacanti et al., 2000, Orthop. Clin. North Am. 31: 351-56). According to the methods of the invention, a tissue explant, such as a microvascular free flap, may be engineered using its native microvasculature as a foundation.

For example, in certain embodiments, a microvascular free flap is used to support the growth of introduced autologous cells, heterogenous cells, pluripotent cells, primordial cells, stem cells, embryonic stem cells, totipotent cells, etc. Such a microvascular free flap used to support the growth of introduced cells may be, in certain embodiments, also genetically transformed as disclosed herein. Such a microvascular free flap, with the introduced cells, may be maintained ex vivo and the introduced cells induced to proliferate and/or differentiate. The microvascular free flap with associated introduced cells is then returned to a patient using standard microvascular techniques, where it subsequently functions as a neo-organ. In preferred embodiments, the recipient of the transplanted neo-organ is also the donor of the microvascular free flap from which the neo-organ is engineered or constructed.

In certain embodiments, the microvascular free flap can be maintained in a bioreactor as described above (see also Niklason et al., 1999, Science 284(5413): 489-493). This permits extensive modifications to be performed using, e.g., transcriptional activating factors, protein growth factors and bioengineered cells, without risk of toxicity to the eventual recipient. A flap may also be modified or engineered to achieve organ level complexity and then used to replace deficient functions (i.e., renal, hepatic, pancreatic, etc.) in the recipient.

In certain embodiments, the vasculature of the flap is expanded through pro-angiogenic stimuli (e.g., hypoxia, vascular mitogen such as VEGF, transplantation of bone-marrow derived endothelial progenitor cells (EPCs) ).

As discussed above, the microvascular free flap may be used to engineer a neo-organ. In such embodiments, a population of introduced cells is “seeded” or established in the microvascular free flap by introducing, e.g., autologous cells, heterogenous cells, pluripotent cells, primordial cells, stem cells, embryonic stem cells, totipotent cells, differentiated cells, etc., into the bioreactor system. A large variety of primordial or stem cells can be isolated according to methods commonly known in the art and can be maintained in association with a flap in an immunodeficient host or in a bioreactor. Such primordial or stem cells include, but are not limited to, embryonic stem cells, mesenchymal stem cells, parenchymal cells, stromal cells, endothelial cells, hepatocytes, keratinocytes, and stem or progenitor cells for a particular cell type, tissue or organ, including but not limited to neurons, myelin, muscle, blood, bone marrow, skin, heart, connective tissue, lung, bronchioles, kidney, liver, and pancreas (e.g., pancreatic islet cells). In certain embodiments, the introduced cells differentiate ex vivo into mature cells, e.g., end-organ cells. In other embodiments, introduced stem cells differentiate ex vivo.

In one embodiment, a microvascular free flap is transformed genetically and maintained in a bioreactor for a period of time, e.g., a day, a week, a month or longer.

In another embodiment, a microvascular free flap is transformed genetically, maintained in a bioreactor for a period of time, e.g., a day, a week, a month or longer, then transplanted into a recipient.

In another embodiment, a microvascular free flap is maintained in a bioreactor for a period of time, e.g., a day, a week, a month or longer, during which time it is engineered with cells as described hereinabove.

In another embodiment, a microvascular free flap is maintained in a bioreactor for a period of time, e.g., a day, a week, a month or longer, during which time it is engineered with cells as described hereinabove, then transplanted into a recipient.

In another embodiment, a microvascular free flap is maintained in a bioreactor for a period of time, e.g., a day, a week, a month or longer, during which time it is engineered with cells and transformed genetically as described hereinabove.

In another embodiment, a microvascular free flap is maintained in a bioreactor for a period of time, e.g., a day, a week, a month or-longer, during which time it is engineered with cells and transformed genetically as described hereinabove, then transplanted into a recipient.

In certain embodiments, an intra- or an extra-vascular route is used to deliver cells of interest to a microvascular bed (FIG. 5), so that the cells, or a subset thereof, adhere and incorporate into the vascular bed and extracellular matrix. In certain embodiments, cells that are delivered via an intra- or extra-vascular route migrate or infiltrate into the microvascular bed. When delivered via an extra-vascular route, they may, in certain embodiments, migrate or infiltrate into the microvascular bed from the environment surrounding the microvascular bed. In another embodiment, pluripotential stem cells and/or differentiated cells are introduced into a microvascular bed, and later, stimulated to differentiate and proliferate, respectively, in situ, thereby increasing the total functional cellular mass.

The bone marrow (BM) contains two easily accessible stem cell populations that may be introduced or delivered to a microvascular free flap according to the methods of the invention: the hematopoietic stem cell (HSC) system and mesenchymal stem cells (MSCs) within the surrounding stroma. MSCs are cells with the ability to differentiate into various tissues such as bone, cartilage, fat, muscle, blood vessels, and nerves, both in vivo and ex vivo (Pittenger et al., 1999, Science 284: 143-7; Prockop et al., 1997, Science 276: 71-4; Kopen et al., 1999, Proc. Natl. Acad. Sci. USA 96: 10711-6). HSC-line age cells, best known for their clinical applications to restore hematopoiesis in cancer patients after chemotherapy or irradiation, also have utility in tissue engineering as they have recently been demonstrated to play a role in neovascularization in a variety of pathologic and physiological processes, and have even been shown to differentiate into hepatocytes (Lagasse et al., 2000, Nature Medicine 6: 1229-34; Shi et al., 1998, Blood 92: 362-7).

Three different populations of bone marrow cells (whole bone marrow, MSCs, and HSCs) can be delivered to the microvascular bed via either an intra and extra-vascular route. While therapeutic bone marrow cell transplantation in vivo is generally limited by their tendency to first home back to the bone marrow and later be recruited for biologic regeneration (Pereira et al., 1995, Proc. Natl. Acad. Sci. USA 92: 4857-61; Wang et al., 2001, J. Thorac. Cardiovasc. Surg. 122: 699-705; Gao et al., 2001, Cells Tissues Organs 169: 12-20), bone marrow-derived stem cells when continuously maintained and perfused within a bioreactor system, are more effectively incorporated in microvascular tissue beds because of the absence of the intermediate bone marrow compartment. In addition to stem cells, in certain embodiments differentiated cells may be used and engrafted in the scaffold of the microvasculature.

In another embodiment, endothelial progenitor cells (EPCs) derived from bone marrow are introduced. EPCs are known to participate in postnatal neovascularization and to contribute quantitatively to newly formed vascular structures. Under ex vivo maintenance conditions as described herein, and in association with the explanted flap, the EPCs differentiate into an expanded vasculature in association with the flap. Such a flap with an expanded vasculature can, in certain embodiments, undergo further modification by genetic transformation or by tissue engineering according to the methods of the invention, then reimplanted into a recipient.

5.3. Methods of Tissue Reimplantation into a Host or Recipient

Using conventional surgical procedures (see e.g., Petry et al., 1984, Plast. Reconstr. Surg. 74: 410-33; Blackwell et al., 1997, Head Neck 19, 620-28), the flap is then reinserted into the patient and re-anastomosed to a section of the circulatory system in the patient. Preferably, the flap is attached non-orthotopically, i.e., it is re-anastomosed to a different area of the patient's circulatory system. For example, a flap may be detached from its supply from the femoral artery, transfected by perfusion, then transplanted to the region of the carotid artery and attached to the carotid arterial system. In another embodiment, the flap is reattached to the blood vessels from which it was excised. Preferably, a splint or other protective device is placed over the operative site after attachment or reanastomosis.

In one aspect of the invention, the selected tissue is transplanted to effect chronic or continuous secretion of bioactive molecules into the circulatory system. In another aspect, the tissue is transplanted to effect temporary or finite duration secretion of the bioactive molecule into the circulatory system. In yet another aspect, the tissue is transplanted to effect non-systemic or localized expression of a protein or product of interest.

Following transfection, flaps can be flushed with perfusion medium (e.g., University of Wisconsin (UW) medium). The variables that can be examined include temperature of the flush (4° C., 20° C., 37° C.) and volume of flush (5-250 cc) (Franken et al., 1999, Microsurgery 19(5): 214-22). Positive controls can consist of animals in which the vector of interest is injected e.g., via intravenous or intraperitoneal injection (i.e., in vivo) and negative controls can consist of animals that receive no vector in the initial perfusion solution. Animals can be sacrificed and organs can be harvested at 7 days, near the peak of adenoviral expression. Specimens can be examined for presence of a gene of interest or expression thereof, e.g., with X-gal staining, antibody staining, PCR, etc.

PCR is preferably employed on negative specimens to confirm the absence of any viral DNA sequences in tissues other than the flap and recipient bed. Histologic analysis using standard art-known methods can be employed to define any toxicity produced by the flushing protocol. Preferably, the gene product is present in the treated flap and the local recipient bed, but not in any of the other tissues examined from the recipient.

In certain cases, re-implantation of the microvascular free flap may produce a substantial degree of scarring, thus obscuring the viability of the tissue independent from surrounding tissue. If this occurs, methods commonly known in the art, such as separation with silicone sheets, may be utilized to separate a re-implanted microvascular free flap from the host in order to prevent tissue ingrowth.

5.4. Tissues and Organ Systems for Transfection

According to the methods of the invention, explanted microvascular free flaps (or beds) are transfected ex vivo. The microvascular free flaps can comprise tissue that includes, but is not limited to, epithelial tissues, e.g., the epidermis, gastrointestinal tissue; connective tissues, e.g., dermis, tendons, ligaments, cartilage, bone and fat tissues, blood; muscle tissues, e.g., heart and skeletal muscles; nerve tissue, e.g., neurons and glial cells. The microvascular free flaps or beds can also comprise tissue derived from organs or organ systems such as the skeletal system, e.g., bones, cartilage, tendons and ligaments; the muscular system, e.g., smooth and skeletal muscles; the circulatory system, e.g., heart, blood vessels, endothelial cells; the nervous system, e.g., brain, spinal cord and peripheral nerves; the respiratory system, e.g., nose, trachea and lungs; the digestive system, e.g., mouth, esophagus, stomach, small and large intestines; the excretory system, e.g., kidneys, ureters, bladder and urethra; the endocrine system, e.g., hypothalamus, pituitary, thyroid, pancreas and adrenal glands; the reproductive system, e.g., ovaries, oviducts, uterus, vagina, mammary glands, testes, seminal vesicles and penis; the lymphatic and immune systems, e.g., lymph, lymph nodes and vessels, white blood cells, bone marrow, T- and B-cells, macrophage/monocytes, adipoctyes, keratinocytes, pericytes, and reticular cells.

In certain embodiments, the selected tissue is autologous. In other embodiments, the tissue is heterogenous.

5.5. Bioactive Molecules

Using the methods of the invention, selected tissue is genetically transformed ex vivo, then reimplanted in a patient in need of the transformed tissue. Numerous nucleic acids may be used for such transformation, including, but not limited to, the nucleic acids encoding the bioactive molecules presented in Table 1.

TABLE 1
Bioactive Molecules
Classical Growth Factors

VEGF Family
VEGF-A
VEGF-B
VEGF-C
VEGF-D
Placental Growth Factor
Angiopoietin-1
Angiopoietin-2
Angiopoietin-3
Angiopoietin-4
VEGFR-1
VEGFR-2
VEGFR-3
Tie-1
Tie-2 (Tek)
EGF Family
TGF-a
EGF
HB-EGF
Heregulin
ErbB-3
ErbB-4
Neu
EGF-R
PDGF Family
PDGF-A chain
PDGF-B chain
PDGFR-alpha
PDGFR-beta
TGF-beta Family
TGF-b1
TGF-b2
TGF-b3
Activin
Follistatin
BMP1
BMP-2
BMP-3
BMP-4
BMP-5
BMP-6
BMP-7
BMP-8
GDF-1
GDF-2
GDF-3
GDF-4
GDF-5
GDF-6
GDF-7
GDF-8
GDF-9
Inhibin alpha
Inhibin beta
TGF-b receptor I
TGF-b receptor II
TGF-b receptor III
Alk-1
Alk-2
Alk-3
Alk-4
Alk-5
Alk-6
SMAD1
SMAD2
SMAD3
SMAD4
SMAD5
SMAD6
SMAD7
SMAD8
SMAD9
TGIF
Nodal
Noggin
Osteocalcin
NGF Family
NGF
BDNF
Trk A
Trk B
Trk C
NGFR p75
CNTF
CNTFR
GDNF
GFRalpha-1
GFRalpha-2
GFRalpha-3
Neuropilin
Presenilin 1
Presenilin 2
FGF Family
FGF-1
FGF-2
FGF-3
FGF-4
FGF-5
FGF-6
FGF-7
FGF-8
FGF-9
FGF-10
FGF-11
FGF-12
FGF-13
FGF-14
FGF-15
FGF-16
FGF-17
FGF-18
FGF-19
FGF-20
FGFR-1
FGFR-2
FGFR-3
FGFR-4
IGF Family
IGF-I
IGF-II
IGFBP-1
IGFBP-2
IGFBP-3
IGFBP-4
IGFBP-5
IGFBP-6
IGFBP-7
IGF-IR alpha subunit
IGF-IR beta subunit
HGF Family
HGF alpha
HGF beta
HGFL (MSP)
Met
TNF Family
TNF-a
TNF-beta
TNF-R1
TNF-R2
CTGF Family
CTGF
CTGF receptor
CSF Family
G-CSF
GM-CSF
M-CSF
CSF-1R
G-CSFR

Interferons

IFN-alpha
IFN-beta
IFN-gamma

Interleukins

IL-1a
IL-1b
IL-2
IL-3
IL-4
IL-5
IL-6
IL-7
IL-8
IL-9
IL-10
IL-11
IL-12a
IL-12b
IL-13
IL-15
IL-16
IL-17
IL-18

Morphogens

Wnt Family
Wnt-1
Wnt-2a/Wnt-13
Wnt-2b
Wnt-3a
Wnt-4
Wnt-5a
Wnt-5b
Wnt-6
Wnt-7a
Wnt-7b
Wnt-8a
Wnt-8b
Wnt-1Oa
Wnt-1Ob
Wnt-11
Wnt-14
Wnt-15
Wnt-16
Fzd1
Fzd2
Fzd3
Fzd4
Fzd5
Fzd6
Fzd7
Fzd8
Fzd9
Fzd10
Frp-1
Frp-2
Frp-3
Frp-4
Frp-5
B-catenin
APC
APC2
TCF-1
TCF-2
TCF-3
LEF-1
Dishevelled-1
Dishevelled-2
Dishevelled-3
Axin
Hedgehog Family
SHH
DHH
IHH
Patched
Smoothened
Gli1
Gli2
Gli3
Notch Family
Notch 1
Notch 2
Notch 3
Notch 4
Delta
DLK
Jagged
Id Family
Id1
Id2
Id3
Id4
Myo Family
Myo-D
Myogenin
Musculin
Myf-5
Myf-6
Twist
Homeobox genes
Pax-1
Pax-2
Pax-3
Pax-4
Pax-5
Pax-6
Pax-7
Pax-8
Pax-9
HoxA1
HoxA2
HoxA3
HoxA4
HoxA5
HoxA6
HoxA7
HoxA8
HoxA9
HoxA10
HoxA11
HoxA12
HoxB1
HoxB2
HoxB3
HoxB4
HoxB5
HoxB6
HoxB7
HoxB8
HoxB9
HoxC4
HoxC5
HoxC6
HoxC7
HoxC8
HoxC9
HoxC10
HoxC11
HoxC12
HoxC13
HoxD1
HoxD3
HoxD4
HoxD8
HoxD9
HoxD10
HoxD11
HoxD12
HoxD13
Sox family
Pbx 1
Pbx 2
Pbx 3
Six1
Six2
Six3
Six4

Classical Hormones

Pituitary Hormones
TSH
FSH
Prolactin
Lutropin
Somatostatin
Peripheral Hormones
Insulin
Insulin fragments
GH
Thyroid hormones
PTH
Calcitonin
Erythropoietin
thrombopoietin
LIF
SCF
Glucagon
Gastrin
CCK
Somatostatin
Leptin
Leptin receptor
HCG

Steroids and Receptors

Estrogens
Progesterones
Estrogen and progesterone receptors
Androgen receptors
Glucocorticoids/receptors
RAR family
RXR family
Thyroid receptors

Cell Growth Molecules

Tumor Suppressors
Rb
P107
P130
P53
P63
P73
MDM2
DCC
BRCA1
BRCA2
GADD family
NF1
NF2
PTEN
VHL
Elongin
Cyclins
Cyclin A family
Cyclin B family
Cyclin C
Cyclin D family
Cyclin E family
Cyclin F
Cyclin G family
Cyclin H
Cyclin I
Cyclin T family
Cdc family
Cdk family
Apoptosis Molecules
Fas
Fas ligand
Trail
Tweak
DAXX
RIP
FADD
TRADD
Bcl-2
Bcl-x
Bax
Bad
Bak
SODD
Caspase-1
Caspase-2
Caspase-3
Caspase-4
Caspase-5
Caspase-6
Caspase-7
Caspase-8
Caspase-9
Caspase-10
Caspase-12
Caspase-14
AIF
Miscellaneous Transcription Factors
Fos
Jun
C/EBP family
CBP/p300
NF-1 family
E2F family
Mad/Max family
Myc/myb family
NF-kappaB family
STAT family
Kinases
ERK family
JNK family
MEK family
MEKK family
P42/44 MAPK
Raf family
DAG
Rho/rac
PKC
PKA
ILK
Src
Fak
Crk
Csk
c-abl
Bcr
JAK family
SOCS-1
SOCS-2
SOCS-3
SOCS-4
SOCS-5
SOCS-6
SOCS-7
Matrix Molecules
ECM Molecules
Collagen I
Collagen II
Collagen III
Collagen IV
Fibronectin
Elastin
Laminin-a1
Laminin a2
Laminin b1
Laminin b2
Laminin b3
Laminin gamma-1
Laminin gamma-2
Laminin-5
Vitronectin
Thrombospondin-1
Thrombospondin-2
Thrombospondin-3
Thrombospondin-4
Syndecan-1
Syndecan-2
Syndecan-3
Syndecan-4
L-selectin
P-selectin
E-selectin
ECM enzymes
MMP-1
MMP-2
MMP-3
MMP-7
MMP-8
MMP-9
MMP-10
MMP11
MMP-12
MMP-13
MMP-14
MMP-15
MMP-16
MMP-17
MMP-19
MMP-20
Plasminogens
Plasminogen activators
PAI-1
PAI-2
Angiostatin
Endostatin
Elastase
Cathepsin B
Cathepsin D
Cathepsin E
Cathepsin F
Cathepsin G
Cathepsin H
Cathepsin K
Cathepsin O
Cathepsin L
Cell Surface Receptors/associated molecules
Integrin a1
Integrin a2
Integrin a3
Integrin a4
Integrin a5
Integrin a6
Integrin aE
Integrin aL
Integrin aM
Integrin aV
Integrin aX
Integrin aIIb
Integrin b1
Integrin b2
Integrin b3
Integrin b4
Integrin b5
Integrin b6
Integrin b7
Integrin b8
Ephrins
VWF
Keratin K12
Keratin type II
Cytokeratin
Keratin type I
Keratin D
Keratin K12
Neurofilament protein
NF-66
Cytokeratin 13
Keratin 16
Lamin B
Hair keratin basic 5
Hair keratin acidic 5
Lamin b3
Cytokeratin 15
Lamin A
Lamin C
Lamin B1
Keratin 6 alpha
Keratin 6 beta
Cytokeratin 17
Type II cytokeratin
LDL receptors
HDL receptors
VLDL receptors

Neurologic Proteins

GABA Receptors
GABA neurotransmitters
NMDA receptors
NMDA neurotransmitters
Dopamine receptors
Dopamine
Muscarinic acetylcholine receptors
Nicotinic acetylcholine receptors
acetylcholine
CRF family
CRF receptor family
Serotonin receptors
serotonin
Opioid receptors
Tyrosine hydroxylase
Dopamine hydroxylase
Substance P

Miscellaneous

Ubiquitin
HIF-1alpha
HIF-2 alpha
HIF-3 alpha
CFTR
TRF family
Telomerases TP1
Telomerase TRT
MHC family molecules
NOS1
NOS2
NOS3
GLUT1
GLUT2
GLUT3
GLUT4
Heme oxygenase 1
Heme oxygenase 2
Catalase
Superoxide dismutase
RAGE (receptor for advanced glycosylation endproducts)

Antimicrobial Peptides

Lysostaphin
PR-39
Magainin 1
Magainin 2
Alpha-Defensin
Beta-defensin
BPI
PLA2
Bombinins
Brevinin-1
Brevinin-1E
Cathelicidins
Cecropins
Histatin I
Protegrins
NK-lysin
Androopin
BLP-1
Bombinin
Cecropin A
Cecropin B
Ceratotoxin
Clavanin
Dermaseptin b
Dermaseptin s
Enbocin
Lycotoxin
Melittin
Misgurin
PGLa
Pleurocidin
Seminalplasmin
Styelin
Abaecin
Apidaecin
Bactenicin
Diptericin
Drosocin
Enkelytin
Formaecin
Indolicidin
Lebocin
Prophenin
Tenecin
Bovine dodecapeptide
Pipinin
Ranalexin
Thanatin
Androctonin
Polyphemusin
Protegrin I
Protegrin 2
Protegrin 3
Cryptdin 1
Cryptdin 5
RK-1
Big defensin
Gallinacin I
LAP
Tracheal antimicrobial peptide
Defensin
Defensin 4K
Formicin A
Royalisin
Sapecin
Drosomycin
Tachycitin
NP-1
NP-2
NP-3A
NP-3B
NP-4
NP-5
HNP-1
HNP-2
HNP-3
Lactoferricin-B
Tritrptcin
Antifungal peptide
1901-II
1907-VIII
Aculeacins
Aureobasidin A
Bacillomycin F
CB-1
Cepacidin A1
Cepacidin A2
Echinocandin B
Fungicin M-4
Helioferin A
Helioferin B
FR900403
Iturin A
Leucinostatin A
Leucinostatin H
Leucinostatin K
Mulundocandin
Nikkomycin X
Niccomycin Z
Pneumocandin
Polyoxin D
Syringomycin E
Syringostatin A
Syringotxin B
Trichpolyn A
Trichopolyn B
WF11899 A
WF11899 B
WF11899 C
Zeamatin

Heat Shock Protein family

Agp-1
Agp-2
GRP 75
GRP 78
GRP 94
HSC 70
HSP 27
HSP 40
HSP 47
HSP 56
HSP 60
HSP 70
HSP 90
HSP 105

Hematologic Molecules

Factor V
Factor VIII
Protein S
Protein C

Chemokines

C-X-C Family
CXCR1
CXCR2
CXCR3
CXCR4
CXCR5
IL-8
Nap-2
Gro-a
Gro-b
Gro-g
ENA-78
SDF-1
IP-10
MIG
I-TAC
SDF-1
BCA-1
GCP-2
KC/CINC-1
MIP-2a
MIP-2b
Platelet basic protein
PF-4
Mig
BLC
WECHE
C-C Family
CCR1
CCR2a
CCR2b
CCR3
CCR4
CCR5
CCR6
CCR7
CCR8
Bonzo
MCP-1
MCP-2
MCP-3
MCP-4
MCP-5
MDC
TARC
Eotaxin-1
Eotaxin-2
HCC-1
HCC-4
C-10
MIP-1a
MIP-1b
MIP-1g
MIP-3a
MIP-3b
MIP-4
MIP-5
6Ckine
CCl27
MPIF-1
SLC
I-309
RANTES
TARC
TECK
C Family
XCR1
Lymphotactin
CX3C Family
CX3CR
Neurotactin (fractalkine)

5.6. DNA

The methods of the invention for delivering a nucleic acid encoding a product of interest can employ a variety of different types of DNA molecules. The DNA molecules may include genomic, cDNAs, single stranded DNA, double stranded DNA, triple stranded DNA, oligonucleotides and Z-DNA.

The DNA molecules may code for a variety of bioactive molecules including extracellular, cell surface, and intracellular RNAs and proteins. Examples of extracellular proteins include growth factors, cytokines therapeutic proteins, hormones and peptide fragments of hormones, inhibitors of cytokines, peptide growth and differentiation factors, interleukins, chemokines, interferons, colony stimulating factors and angiogenic factors. Examples of such bioactive molecules include, but are not limited to, the bioactive molecules are presented in Table 1 (above).

The DNA molecules may also code for proteins that block pathological processes. Examples of blocking factors include ribozymes that destroy RNA function and DNAs that, for example, code for tissue inhibitors of enzymes that destroy tissue integrity, e.g., inhibitors of metalloproteinases associated with arthritis.

One may obtain the DNA segment encoding the product (protein) of interest using a variety of molecular biological techniques, generally known to-those skilled in the art. For example, cDNA or genomic libraries may be screened using primers or probes with sequences based on the known nucleotide sequences. Polymerase chain reaction (PCR) may also be used to generate the DNA fragment encoding the protein of interest. Alternatively, the DNA fragment may be obtained from a commercial source.

DNA or nucleic acids with sequences that vary from those described in the literature are also encompassed by the invention, so long as the altered or modified nucleic acid still encodes a bioactive molecule of interest that functions in any direct or indirect manner. These sequences include those caused by point mutations, those due to the degeneracies of the genetic code or naturally occurring allelic variants, and further modifications that have been introduced by genetic engineering, i.e., by the hand of man.

Techniques for introducing changes in nucleotide sequences that are designed to alter the functional properties of the encoded proteins or polypeptides are well known in the art. Such modifications include the deletion, insertion or substitution of bases that result in changes in the amino acid sequence. Changes may be made to increase the activity of an encoded protein, to increase its biological stability or half-life, to change its glycosylation pattern, confer temperature sensitivity or to alter the expression pattern of the protein and the like. All such modifications to the nucleotide sequences are encompassed by this invention.

5.7. Vectors

The DNA encoding the translational or transcriptional products of interest may be recombinantly engineered into a variety of vector systems that provide for replication of the DNA in large scale for the preparation of gene activated matrices. These vectors can be designed to contain the necessary elements for directing the transcription and/or translation of the DNA sequence taken up by the tissue ex vivo. Vectors that may be used include, but are not limited to, those derived from recombinant bacteriophage DNA, plasmid DNA or cosmid DNA. For example, plasmid vectors such as pBR322, pUC 19/18, pUC 118, 119 and the M13 mp series of vectors may be used. Bacteriophage vectors may include λgt10, λgt11, λgt18-23, λZAP/R and the EMBL series of bacteriophage vectors. Cosmid vectors that may be utilized include, but are not limited to, pJB8, pCV 103, pCV 107, pCV 108, pTM, pMCS, pNNL, pHSG274, COS202, COS203, pWE15, pWE16 and the charomid 9 series of vectors. Vectors that allow for the in vitro transcription of RNA, such as SP6 vectors, may also be used to produce large quantities of RNA that may be incorporated into matrices. Alternatively, recombinant virus vectors including, but not limited to those derived from viruses such as herpes virus, retroviruses, vaccinia viruses, adenoviruses, adeno-associated viruses (AAV), lentiviruses or bovine papilloma virus may be engineered. Non-viral vectors, such as liposomes, may also be engineered.

A viral vector is preferably chosen that has very high transfection efficiency. Such efficiency is not routinely achieved with plasmid vectors or naked DNA. A retrovirus does not infect nondividing cells, and most of the cells in a microvascular free flap tissue are not dividing. Adenoviral vectors are commonly used for this reason: they achieve high levels of expression since they are able to infect both dividing and nondividing cells through the CAR receptors present on both human and rodent cells.

Unlike the adeno-associated viral vectors (AAV), however, adenoviruses are highly immunogenic and thereby are eventually disposed of by the host. This accounts for the transient expression seen in vivo. This transient, limited expression (7-42 days in most cases) may be of benefit in some situations, but is not preferred following oncologic surgery, where a high local expression of the anti-tumor agent is necessary for several months in order to minimize recurrence. In such cases, AAV vectors may be used, as they are tolerated to a much greater degree than adenoviral vectors, and have demonstrated expression of their genetic packages for up to a year (or longer) following administration in an immunocompetent animal (Jung et al., 2001, Proc. Natl. Acad. Sci. USA 98: 2676-2681). In certain embodiments, an AAV vector is used because of the-ease in inserting the nucleic acid construct of interest into the AAV backbone.

In a preferred embodiment, a therapeutic gene of interest exhibits sustained expression in a microvascular free flap, a property that is generally not possible using adenoviral vectors. Thus, a liposomal transfection (lipofection) method, which is commonly known in the art, can be used. Alternatively, transfection using adeno-associated virus (AAV) vectors may be used, as discussed above. Both methods appear to produce prolonged (and possibly permanent) gene expression.

Methods that are well known to those skilled in the art can be used to construct expression vectors containing the protein coding sequence operatively associated with appropriate transcriptional/translational control signals. These methods include in vitro recombinant DNA techniques, and synthetic techniques. See, for example, the techniques described in Sambrook et al., 2001, Molecular Cloning, A Laboratory Manual, Third Edition, Cold Spring Harbor Laboratory Press, N.Y.; Ausubel et al., 1989, Current Protocols in Molecular Biology, Green Publishing Associates and Wiley Interscience, N.Y., both of which are incorporated by reference herein in their entireties.

The nucleic acids encoding the proteins of interest may be operatively associated with a variety of different promoter/enhancer elements. The expression elements of these vectors may vary in their strength and specificities. Depending on the host/vector system utilized, any one of a number of suitable transcription and translation elements may be used. The promoter may be in the form of the promoter that is naturally associated with the nucleic acid encoding the product of interest. Alternatively, the DNA may be positioned under the control of a recombinant or heterologous promoter, i.e., a promoter that is not normally associated with that nucleic acid. For example, tissue specific promoter/enhancer elements may be used to regulate the expression of the transferred DNA in specific cell types. Examples of transcriptional control regions that exhibit tissue specificity that have been described and could be used, include but are-not limited to: elastase I gene control region, which is active in pancreatic acinar cells (Swift et al., 1984, Cell 38:639-646.; Ornitz et al., 1986, Cold Spring Harbor Symp. Quant. Biol. 50:399-409; MacDonald, 1987, Hepatology 7:42S-51S); insulin gene control region, which is active in pancreatic beta cells (Hanahan, 1985, Nature 315:115-122); immunoglobulin gene control region, which is active in lymphoid cells (Grosschedl et al., 1984, Cell 38:647-658; Adams et al., 1985, Nature 318:533-538; Alexander et al., 1987, Mol. Cell. Biol. 7:1436-1444): albumin gene control region, which is active in liver (Pinkert et al., 1987, Genes and Devel. 1:268-276) alpha-fetoprotein gene control region, which is active in liver (Krunlauf et al., 1985, Mol. Cell. Biol. 5:1639-1648; Hammer et al., 1987, Science 235:53-58); alpha-1-antitrypsin gene control region, which is active in liver (Kelsey et al., 1987, Genes and Devel. 1:161-171); beta-globin gene control region, which is active in myeloid cells (Magram et al., 1985, Nature 315:338-340; Kollias et al., 1986, Cell 46:89-94); myelin basic protein gene control region, which is active in oligodendrocyte cells in the brain (Readhead et al., 1987, Cell 48:703-712); myosin light chain-2 gene control region, which is active in skeletal muscle (Shani, 1985, Nature 314:283-286); and gonadotropic releasing hormone gene control region, which is active in the hypothalamus (Mason et al., 1986, Science 234:1372-1378). Promoters isolated from the genome of viruses that grow in mammalian cells, (e.g., RSV, vaccinia virus 7.5K, SV40, HSV, adenoviruses MLP, MMTV LTR and CMV promoters) may be used, as well as promoters produced by recombinant DNA or synthetic techniques.

In preferred embodiments, the promoter elements are constitutive or inducible promoters and can be used under the appropriate conditions to direct high level or regulated expression of the nucleic acid encoding the product of interest. Expression of a nucleic acid encoding a product of interest that is under the control of a constitutive promoter does not require the presence of a specific substrate to induce expression of the nucleic acid and will occur under all conditions of cell growth. In contrast, expression of a nucleic acid encoding a product of interest controlled by an inducible promoter is responsive to the presence or absence of an inducing agent.

In one embodiment, the nucleic acid encoding the product of interest is expressed conditionally, using any type of inducible or repressible system-available for conditional expression of a nucleic acid encoding a product of interest known in the art, e.g., a system inducible or repressible by tetracycline (“tet system”); doxycycline; interferon; estrogen, ecdysone, or other steroid inducible system; Lac operator, progesterone antagonist RU486, or rapamycin (FK506).

In other embodiments, the promoter elements are endothelial cell-specific, e.g., flk-1(Patterson et al., 1995, J. Biol. Chem. 270: 23111-18); flt-1 (Morishita et al., 1995, J. Biol. Chem. 270:.27948-53); tie-2 (Schlaeger et al., 1995, Development 121: 1089-98); von Willebrand factor (Aird et al., 1997, Proc. Natl. Acad. Sci. USA 92: 4567-71); endothelin-1 (Paul et al., 1995, Hypertension 25:683-693) (for review, see, e.g., Garlanda et al., 1997, Arterioscler. Thromb. Vasc. Biol. 17(7):1193-1202).

Specific initiation signals are also required for sufficient translation of inserted protein coding sequences. These signals include the ATG initiation codon and adjacent sequences. In cases where the entire coding sequence, including the initiation codon and adjacent sequences are inserted into the appropriate expression vectors, no additional translational control signals may be needed. However, in cases where only a portion of the coding sequence is inserted, exogenous translational control signals, including the ATG initiation codon must be provided. Furthermore, the initiation codon must be in phase with the reading frame of the protein coding sequences to ensure translation of the entire insert. These exogenous translational control signals and initiation codons can be of a variety of origins, both natural and synthetic. The efficiency and control of expression may be enhanced by the inclusion of transcription attenuation sequences, enhancer elements, etc.

In addition to DNA sequences encoding therapeutic proteins of interest, the scope of the present invention includes the use of ribozymes or antisense DNA molecules that may be transferred into the mammalian repair cells. Such ribozymes and antisense molecules may be used to inhibit the translation of RNA encoding proteins of genes that regulate a disease process.

The expression of antisense RNA molecules will act to directly block the translation of mRNA by binding to targeted mRNA and preventing protein translation. The expression of ribozymes, which are enzymatic RNA molecules capable of catalyzing the specific cleavage of RNA may also be used to block protein translation. The mechanism of ribozyme action involves sequence specific hybridization of the ribozyme molecule to complementary target RNA, followed by a endonucleolytic cleavage. Within the scope of the invention are engineered hammerhead motif ribozyme molecules that specifically and efficiently catalyze endonucleolytic cleavage of RNA sequences. RNA molecules may be generated by transcription of DNA sequences encoding the RNA molecule.

It is also within the scope of the invention that multiple nucleic acids, combined on a single genetic construct under control of one or more promoters, or prepared as separate constructs of the same or different types may be used. Thus, an almost endless combination of different nucleic acids and genetic constructs may be employed. Certain combinations of nucleic acids in genetic constructs may be designed to, or their use may otherwise result in, achieving synergistic effects on cell stimulation and regeneration; any and all such combinations are intended to fall within the scope of the present invention. Indeed, many synergistic effects have been described in the scientific literature, so that one of ordinary skill in the art would readily be able to identify likely synergistic combinations of nucleic acids, or even nucleic acid-protein combinations.

5.8. Uses for Microvascular Free Flaps

The compositions and methods of the invention for ex vivo modification of tissue by genetic transformation or by tissue engineering can be applied to the treatment of a large number of disorders. In one aspect, the compositions and methods of the invention are used to treat a malignancy or related disorder, including, but not limited to the malignancies and related disorders presented in Table 2.

TABLE 2
MALIGNANCIES AND RELATED DISORDERS

Leukemia

acute leukemia

acute lymphocytic leukemia
acute myelocytic leukemia

myeloblastic
promyelocytic
myelomonocytic
monocytic
erythroleukemia

chronic leukemia

chronic myelocytic (granulocytic) leukemia
chronic lymphocytic leukemia

Polycythemia vera
Lymphoma

Hodgkin's disease
non-Hodgkin's disease

Multiple myeloma
Waldenström's macroglobulinemia
Heavy chain disease
Solid tumors

sarcomas and carcinomas

fibrosarcoma
myxosarcoma
liposarcoma
chondrosarcoma
osteogenic sarcoma
chordoma
angiosarcoma
endotheliosarcoma
lymphangiosarcoma
lymphangioendotheliosarcoma
synovioma
mesothelioma
Ewing's tumor
leiomyosarcoma
rhabdomyosarcoma
colon carcinoma
pancreatic cancer
breast cancer
ovarian cancer
prostate cancer
squamous cell carcinoma
basal cell carcinoma
adenocarcinoma
sweat gland carcinoma
sebaceous gland carcinoma
papillary carcinoma
papillary adenocarcinomas
cystadenocarcinoma
medullary carcinoma
bronchogenic carcinoma
renal cell carcinoma
hepatoma
bile duct carcinoma
choriocarcinoma
seminoma
embryonal carcinoma
Wilms' tumor
cervical cancer
uterine cancer
testicular tumor
lung carcinoma
small cell lung carcinoma
bladder carcinoma
epithelial carcinoma
glioma
astrocytoma
medulloblastoma
craniopharyngioma
ependymoma
pinealoma
hemangioblastoma
acoustic neuroma
oligodendroglioma
menangioma
melanoma
neuroblastoma
retinoblastoma

The compositions and methods of the invention may be used to treat any vertebrate species, e.g., mammal, bird, reptile, amphibian, etc. In certain embodiments, the vertebrate is a mammalian species such as mouse, rat, rabbit, guinea pig, dog, cat, pig, sheep, cow, horse, monkey, etc., and in a preferred embodiment, it is a human.

The compositions and methods of the invention are advantageous because a genetically transformed or modified tissue is reimplanted and uses native microvasculature as a foundation, which permits extensive transcriptional modifications to be performed without risk of toxicity to the eventual recipient. Tissue flaps transfected according to the methods of the invention can be used, for example, to deliver antimicrobial peptides to chronic wounds, growth factors to stimulate healing, and to deliver selected bioactive molecules, such as proteins and hormones, to a patient deficient in such proteins or hormones.

In a specific embodiment, a microvascular free flap is transplanted to an area of interest in a recipient so that antimicrobial peptides (i.e., margain-2) are delivered to an area of osteomyelitis or infected foreign body.

In another embodiment, a microvascular free flap being used for reconstruction is used for localized gene therapy (or brachytherapy). Once re-anastamosed, the flap expresses a high local concentration of a protein of interest, either in the microvasculature or in the surrounding tissues (via a paracrine effect).

The compositions and methods of the invention are especially advantageous to effect systemic delivery of bioactive molecules by providing a tissue source that chronically or continuously secretes the molecule into the circulatory system. In one aspect of the invention, the bioactive molecule is a protein or hormone that is missing or defective in the patient, such as insulin, or factor 8 (von Willenbrand factor) or another blood-coagulation factor. Such bioactive molecules include, but are not limited to, the bioactive molecules presented in Table 1.

The compositions and methods of the invention offer the ability of delivering systemic gene therapy for conditions such as hemophilia or diabetes mellitus. Conventional gene therapy has failed in these diseases because it has been unable to produce the sustained, high level, gene expression that is required clinically (Docherty, 1997, Clinical Sci. 92(4): 32 1-30). This is principally because the vectors currently approved for human use in vivo result in either low transfection efficiencies (retrovirus, liposomes) or transient transfection (adenovirus) (Clesham, 1998, Heart 80(4): 313-4). However, an expendable vascular bed as described herein (e.g., omentum, temporoparietal fascia, etc) is removed, modified under optimized, prolonged ex vivo conditions, and this results in a much higher transfection efficiency. Following return to the host, these flaps function to produce the encoded gene product in high quantities and act as biologic pumps, reversing the clinical disease state.

By achieving higher levels of transfection, sufficient protein production may be obtained for a systemic effect. The expendable vascular bed is used as a “protein pump,” releasing deficient hormones or cytokines to correct a systemic deficiency. One application for these methods is in the treatment of Type I diabetes. Other applications for these methods include the treatment of hemophilia, dwarfism, etc. In one embodiment, an adenovirus containing an engineered tetrabasic proinsulin nucleic acid (to hasten intracellular processing) is used in a rodent model of Type I diabetes.

In another embodiment, a flap transfected according to the methods of the invention is used for chronic or continuous delivery of humanized monoclonal antibodies.

In a preferred embodiment, flaps modified according to the methods of the invention are used to deliver nucleic acids encoding a cDNA of interest for oncologic therapy. Free flaps can provide a twofold benefit for cancer patients. First, by solving the clinical/ablative problem via tissue reconstruction and second, by providing localized gene activity for direct therapeutic or prophylactic oncologic purposes. Currently, methods of delivering gene therapy containing cDNAs for several oncolytic factors have shown encouraging results but are limited by toxic side effects and difficulty in local targeting (Lamont et al., 2000, Ann. Surg. Oncol. 7: 588-92; Breau et al., 1996, Curr. Opin. Oncol. 8: 227-31;Li et al., 1999, Clin. Cancer Res. 5: 1551-56; Feldman et al., 2000, Cancer 89: 1181-94; Roth et al., 1997, J. Natl. Cancer Inst. 89: 21-39; Hermiston, 2000, J. Clin. Invest. 105: 1169-72; Heise et al., 2000, J. Clin. Invest. 105: 847-51). The use of microvascular flaps according to the present invention provides the ideal vehicle for localized gene therapy and, as demonstrated in Section 6 below, is feasible with the current generation of viral vectors.

For example, a genetically transformed or modified flap can act as a delivery vehicle for localized gene therapy with demonstrable antitumor effect. A number of antitumor agents may be delivered by a genetically transformed or modified free flap (prodrugs, tumor suppressors, antisense RNA). Preferably, well documented agents such as immune modulatory cytokines, e.g., IL-2, IL-4, IL-12 and interferon-alpha (Huang et al., 1996, Gene Therapy. 3(11): 980-7; Caruso et al., 1996, Proc. Natl. Acad. Sci. USA. 93(21):.11302-6), are delivered.

In one embodiment, a microvascular free flap that is modified according to the methods of the invention is used for “biologic brachytherapy” for tumors, e.g., tumors with high rates of recurrence and/or positive resection margins. These tumors include head and neck malignancies, sarcomas and central nervous malignancies, all of which have profound problems with local tumor control.

In a specific embodiment, a microvascular bed is transfected ex vivo with a vector containing, e.g., interleukin 12 (IL-12) or endostatin, to ameliorate the effects of histiosarcoma.

The compositions and methods exemplified herein have relatively broad and far reaching applications. They represents a crossroads between a physiologic approach to human disease and the unrealized promise of molecular biology to treat human disease.

The non-limiting examples below exemplify applications utilizing this method of ex vivo flap modification by genetic transformation or by modification by introduction of cells.

The following experimental examples are offered by way of illustration and not by way of limitation.

### 6. EXAMPLE 1

### Ex Vivo Transfection of Microvascular Free Flaps

6.1. Introduction

Gene therapy using viral vectors holds great clinical promise but has been limited by difficulties in developing targeted, high-level gene expression with acceptable host toxicity.

The following example demonstrates a method of delivery of a nucleic acid encoding a product of interest that avoids many of the problems associated with viral transfection. Using a rat model, explanted microvascular free flaps were transfected ex vivo, flushed, and reattached to the native circulation using microvascular techniques. A nucleic acid encoding β-galactosidase (β-gal) as a reporter gene was used to demonstrate delivery of a product of interest according to the methods of the invention. Transfection was performed using an adenoviral vector containing the β-galactosidase (β-gal) reporter gene driven by the CMV promoter.

High regional expression of the β-gal gene was seen in the explanted flap in all cell types. No β-gal staining was observed outside of the transfected flap, and almost no viral sequence was detectable by PCR analysis outside of the flap. Further study confirmed that high-level transgene expression was precisely localized to the explanted flap and no collateral transfection occurred in other tissues (liver, spleen, etc.). Currently, human gene therapy requires systemic administration of a nucleic acid (in vivo) or removal of isolated cells for modification (ex vivo) and subsequent re-infusion. The methods of the invention exemplified in this example represent an advance over currently utilized techniques of delivery a nucleic acid and are ideal for targeted gene transfer or targeted delivery of a nucleic acid encoding a product of interest, in patients undergoing microvascular free flap transfers, for example, following oncologic surgery.

6.2. Materials and Methods

Adult male Sprague Dawley rats weighing 250-350 grams were used for this study. Animals were cared for at the animal care facilities of the New York University Medical Center, New York, N.Y. and the M.D. Anderson Cancer Center, Houston, Tex. under standard temperature, humidity and light conditions.

Adenovirus preparation. AdCMVlacZ is a recombinant, replication-deficient adenovirus derived from adenovirus type 5 and was generously donated by Dr. Fang of the MD Anderson Cancer Center. It contains a nuclear-targeted β-galactosidase-encoding nucleic acid driven by the cytomegalovirus (CMV) promoter. The virus was grown by infecting a 293 human embryonic kidney cell line. Following successful infection, the cells were collected, washed and sonicated-to disrupt the membranes. Viral DNA was purified on a CsCl spin gradient and the number of viral particles were determined spectrophotometrically. Titers of virus stocks were determined on an NIH 3T3 plaque assay, and were expressed in PFU/ml. Each viral aliquot was freeze-thawed once.

Anesthesia. Sprague-Dawley rats were anesthetized with an intra-peritoneal cocktail containing Ketamine (150 mg/ml), xylazine (30 mg/1.5 ml) and acepromazine (5 mg/0.5 ml) at a dose of 0.5-0.7 ml/kg. Anesthesia was maintained as needed with a 25% bolus of the initial dose of ketamine alone.

Surgical Procedure/Flap Harvest. Harvest of a superficial epigastric (SE) flap was performed on adult male rats (n=50). The operative procedure resulted in the removal of a 3×2-cm segment of skin and subcutaneous tissue based on the SE vessels in the lower abdomen and groin (Petry et al., 1984, Plast. Reconstr. Surg. 74: 410-33). Clamps were maintained on the proximal femoral vessels while the flap was ex vivo. A 0.2 mm inner diameter infusion catheter (BioTime Micro-cannula, BioTime, Inc., Berkeley, Calif.) was placed in the SE artery and secured with suture. The flap was wrapped in gauze and placed in either cold saline solution for cold ischemia (n=26) or left at room temperature (n=24) (FIG. 1). The flap was perfused, ex vivo, through the arterial catheter, with 20 cc of 1× Dulbecco's PBS (n=40) at a constant perfusion pressure to flush out blood from the flap vessels. Flap infusions were performed at physiologic perfusion pressures between 80-200 mmHg ±5 mmHg to reduce barotrauma from the infusion itself. The infusion rate and pressure were monitored using a Harvard Apparatus pump (Holliston, Mass.) and pressure gauge. During this infusion, the vein was left open to allow outflow of the suspension or solution. In attempts to increase transfection efficiency, we used supranormal pressure infusion (>300 mmHg) using manual delivery, in a group of 10 rats.

In 30 rats, the vein was clamped to occlude outflow and a 5 cc syringe containing (2.5×10⁹ PFU/ml) AdCMVLacZ in PBS was attached to the arterial cannula. 300 μl of AdCMVLacZ was infused into the flap, until the vein was distended, and for the-next 30-60 minutes approximately 300 μl aliquots were intermittently added to the flap to keep the outflow vein continuously distended. The suspension was allowed to dwell in the flap between infusions and for up to one hour after completion. The first 20 animals had infusion of 20 cc of viral suspension without the vein clamped and without-any dwell time. The viral concentration in this initial study was also varied, either 1×10⁶ PFU/ml or 4×10¹¹ PFU/ml. The remainder (n=30) of the rats had the infusion of the virus as described above.

After infusion, the venous occlusion clamp was removed and the flap was flushed again with 10 ml of 1×PBS and the surfaces were rinsed to wash out any free adenovirus. The SE vessels were re-anastomosed to the proximal femoral vessels to establish patency. The flap was re-inset into the groin and a customized, hardened splint was placed over the operative site.

Specimen harvest and analysis. Animals were euthanized by CO₂ narcosis. The flap, contralateral groin skin and samples from the liver and spleen were immediately harvested. Some samples were placed in X-gal development solution (Roche) and then fixed in 4% paraformaldehyde and processed for histologic analysis. Ten high power fields were examined and the total number of each tissue type was counted. The number of blue stained cells and percentages were also calculated.

Assay of Gene Transfer Efficiency. Specimens from all noted areas were also evaluated for lacZ transgene transcript accumulation by means of polymerase chain reaction (PCR). One μg of each RNA sample was reverse transcribed, and 40 PCR cycles were applied to one-tenth of the volume of the initial reverse transcription reaction. The primers were designed to amplify a 404 bp sequence of the bacterial LacZ gene. The sequence of the forward primer was 5′-GATCAAATCTGTCGATCCTTCC-3′ (SEQ ID NO.: 1) and the sequence of the reverse primer was 5′-CAAAGACCAGACCGTTCATACA-3′ (SEQ ID NO.: 2). Amplified DNA fragments were analyzed on 1% agarose gels and amplified bands were visualized under UV transillumination.

Recombinant protein detection. The P-Gal ELISA (Roche) is based on the sandwich ELISA principle. Total protein (TP) was extracted from 5 mm³ homogenized samples with a tissue protein extraction reagent (T-Per, Pierce). TP was measured with an assay (BCA, Pierce) and equal amounts of protein were added in each well of the β-Gal ELISA plate. Ultimately, the absorbance of the sample is determined and is directly correlated to the level of β-Gal present in the cell extract. These readings were compared between transfected flaps at different time points and also to non-flap tissues.

6.3. Results and Discussion

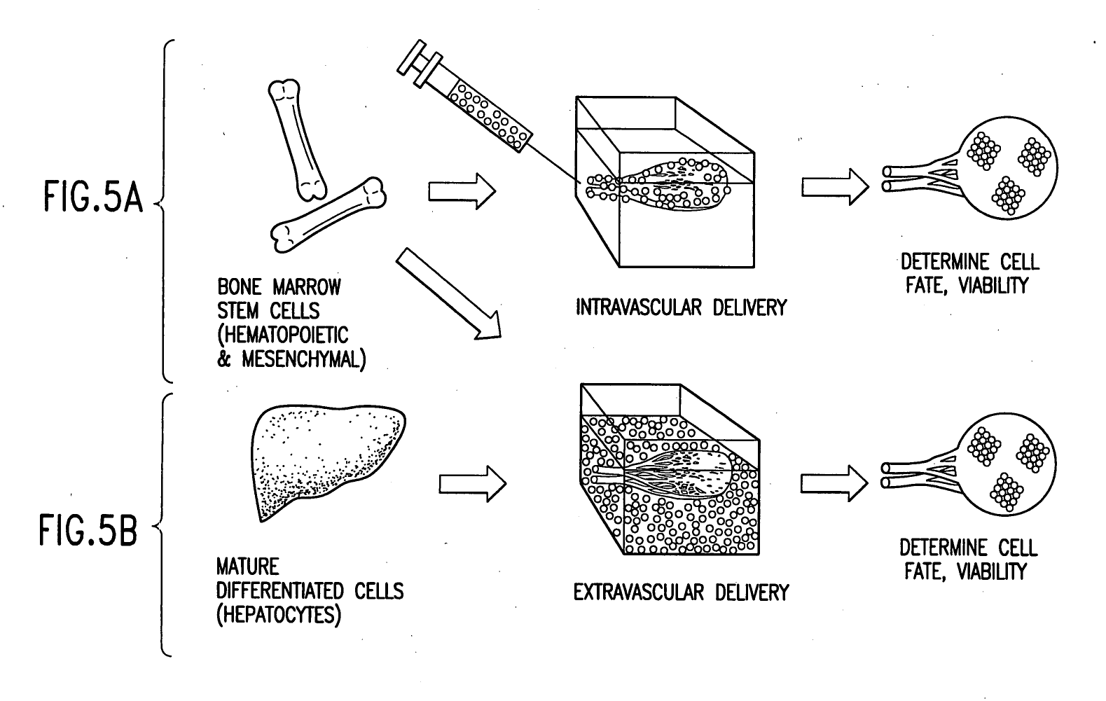

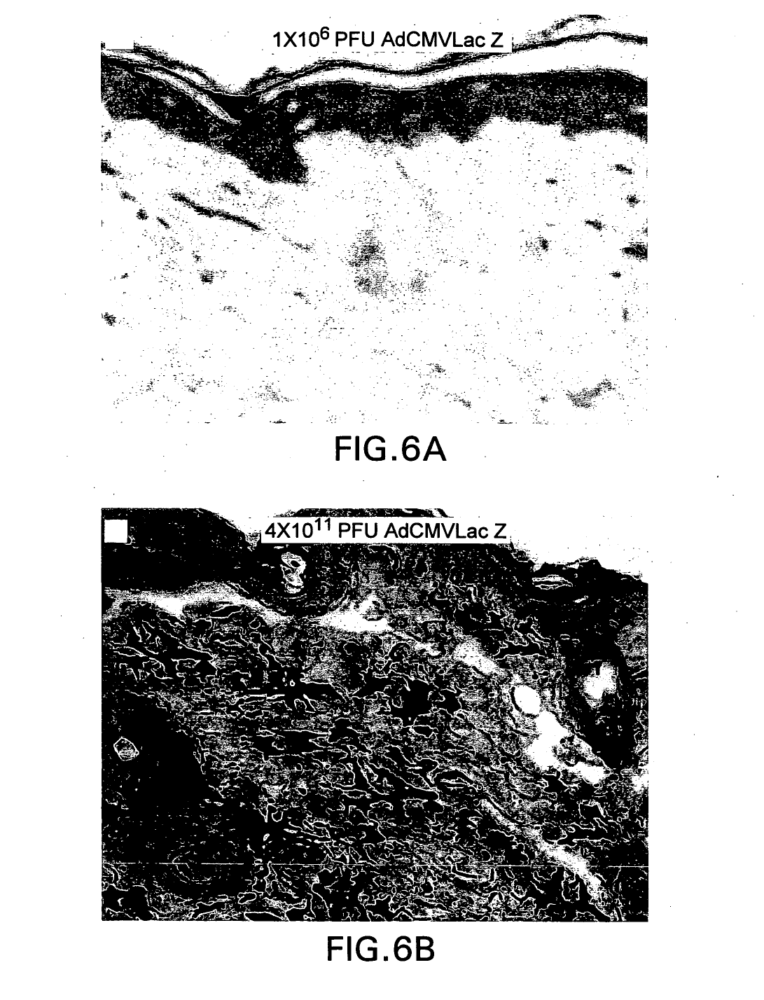

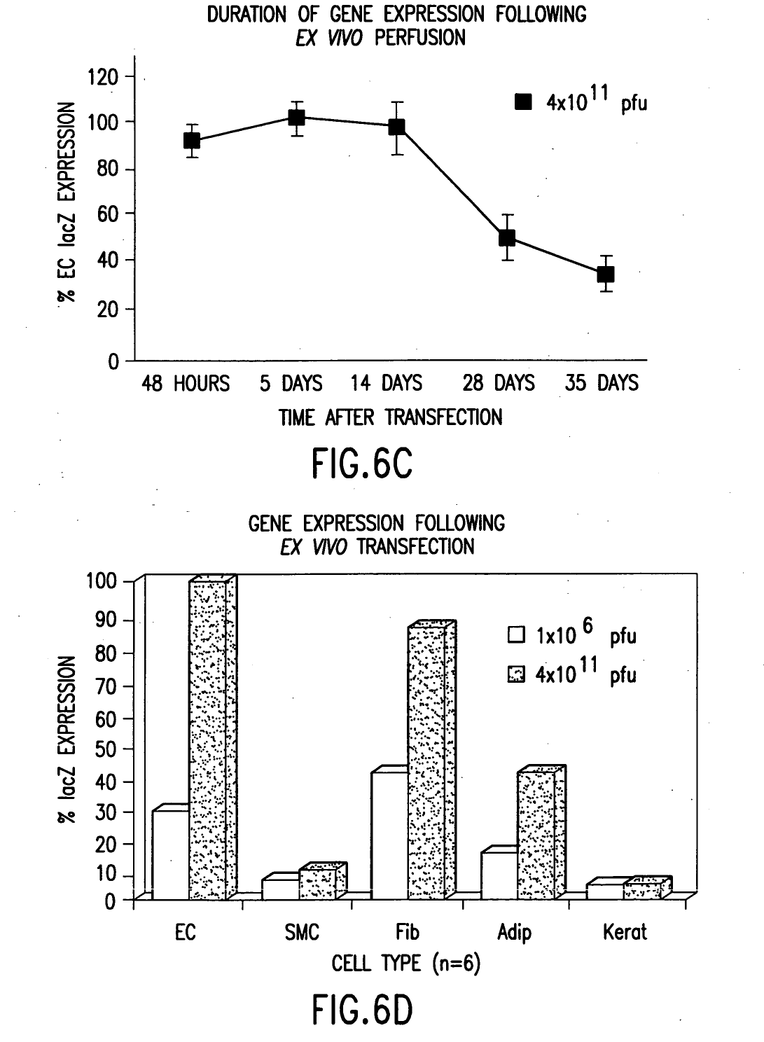

To determine whether free flaps can be transfected ex vivo, we utilized a well-known rat microvascular free flap model based on the superficial epigastric (SE) vessels (FIG. 1B) (Petry et al., 1984, Plast. Reconstr. Surg. 74: 410-33). In initial experiments, we attempted to determine the optimal viral concentration to obtain transfection using twenty adult SE flaps. The SE flaps were infused with twenty ml of an adenoviral vector containing the β-galactosidase (β-gal)-encoding nucleic acid (reporter) driven by the CMV promoter (AdCMVLacZ) at a concentration of either 1×10⁶ plaque forming units/ml (PFU/ml) (10 animals) or 4×10¹¹ PFU/ml (10 animals) through the arterial pedicle without clamping of the venous outflow (FIG. 1C). This was followed by flushing with phosphate buffered saline (PBS) and re-anastomosis (FIG. 1D,E). Animals were sacrificed at 2, 5, 14, 28 and 35 days post-operatively. At each time point specimens were stained for chemical detection of β-Gal (FIGS. 6A and 6B) and cell counts of β-Gal stained cells were performed (FIG. 6C).

Specimens from the lower concentration virus group (1×10⁶ PFU/ml) had barely noticeable levels of transfection for all cell types while the higher concentration group (4×10¹¹ PFU/ml) had excellent β-gal gene transfection at all cell levels with the greatest levels at the 5-14 day time points (FIG. 6D). The transfection levels at these time points on average were, endothelial cells-100%, connective tissue cells-40%, adipocytes-30%, keratinocytes-15% (FIG. 6C). No β-gal staining was noted in non-flap tissues including skin from the contralateral groin, liver and spleen. This was confumed by PCR analysis.

Although we already had excellent transfection levels using the adenoviral vector an effort was made to further improve the transfection efficacy by increasing endothelial permeability with high infusion pressures. Several recent studies had examined the effects of high pressure on viral transfection, and it was these encouraging findings that prompted us to explore supra-normal pressure infusion (Feeley et al., 2000, Transplantation 69: 1067-74; von der Leyen et al., 1999, Hum. Gene Ther. 10: 2355-64). A group of ten flaps received supranormal (>300 mmHg) pressure infusion during the ex vivo period. Animals were sacrificed at post-operative day 5. At this time point, all flaps had areas of partial or full thickness necrosis and there was variable pedicle thrombosis noted on harvest. β-Gal staining was performed on intact specimens and good β-gal reporter gene transfection was noted in the few viable samples, primarily surrounding the larger pedicle vessels. However, there was little to no transfection of adipocytes or connective tissue in these flaps (data not shown). From these experiments it appeared that the flap microcirculation was injured from acute, supranormal intravascular pressure increase, which led to tissue loss and thrombosis.

To further refine targeting to the flap tissues we examined transfection levels when using decreased viral titers. We wanted to more rigorously examine the systemic tissues to see if transfection was isolated to the flap tissue. The initial study, described above, examined differences between high and low viral titer infusion and a dose-response reaction was noted throughout the tissues. From this initial study the viral titer was adjusted to a level of 2.5×10⁹ PFU/ml.

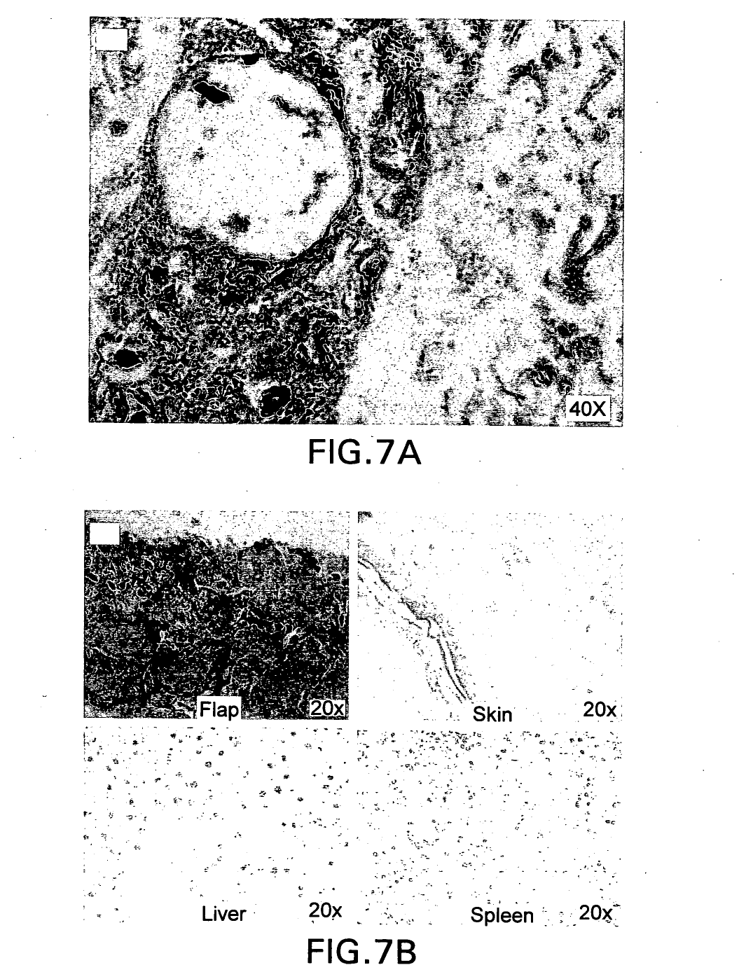

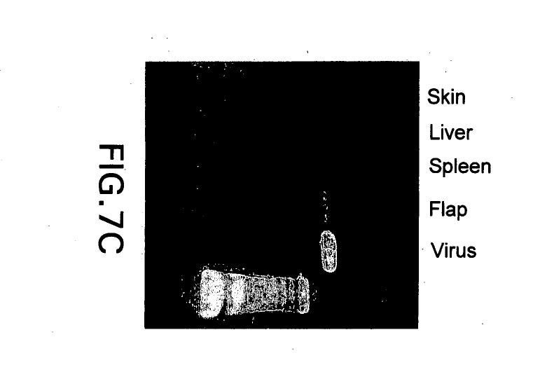

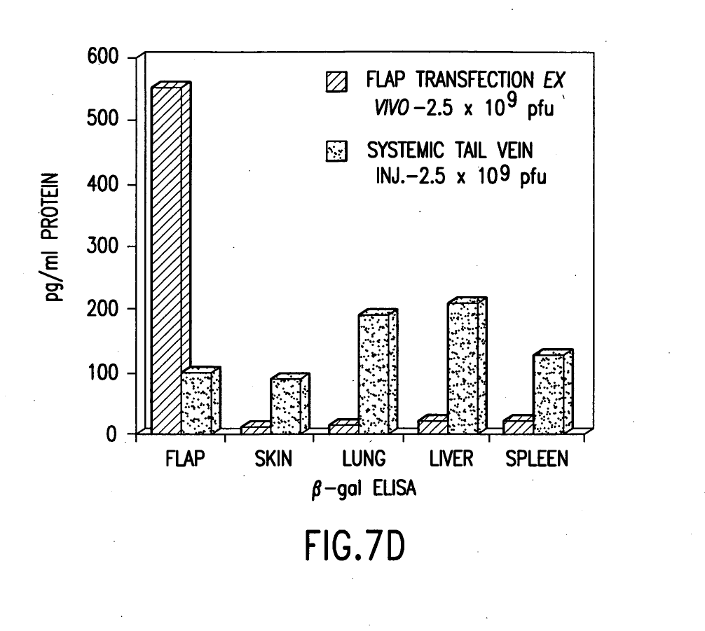

The next 20 rats had 5 nml of AdCMVLacZ infused at a concentration of 2.5×10⁹ PFU/ml while the efferent vein was now continuously clamped. During the viral infusion period the flaps were placed in either cold saline-soaked sponges or at room temperature to evaluate whether any flap viability or transfection differences could be noted. Infusions were given at physiologic pressures (80-200 mm Hg). Physiologic pressure infusion appeared to provide more consistent flap survival and tissue perfusion of the adenovirus. Animals were sacrificed at post-operative days 2, 5, and 15. β-Gal staining was performed and excellent β-Gal gene transfection was noted at all tissue levels within the flap. No significant β-Gal staining was noted in non-flap tissue specimens (FIGS. 7A and 7B).

These specimens were also analyzed using PCR and no significant PCR signals were noted in distant tissues indicating that viral targeting was limited to the flap (FIG. 7C). A β-Gal ELISA was also performed to give a quantitative determination of β-Gal protein levels in tissue, and the results were consistent with the histologic findings as far as total β-Gal activity (FIG. 7D).

The results showed increased activity from the 2 to 5 day specimens with a slight drop off in the 15 day specimens, again consistent with the histologic specimens. No significant β-Gal levels were noted in distant tissues on ELISA. There did not appear to be a difference in transfection efficiency between cold and warm ischemia. This may be because the ex vivo period was limited to less than two hours.

In this portion of the experiment, the viral perfusion conditions were varied slightly from the initial study where the vein was left open and a larger volume of virus was infused. In this study, smaller viral volumes were allowed to dwell with the vein clamped. We were able to obtain excellent transfection levels using either technique.

To compare targeting differences with in vivo as opposed to ex vivo flap transfection, we injected an identical viral bolus through the tail vein at the same concentration. The same tissues were harvested and compared to similar experimental time points using the β-Gal ELISA (FIG. 7C). The systemic injection group had broad, low level β-Gal activity. Conversely, the flap group, β-Gal activity was much higher in the transfected flap than any other systemic tissue. This result confirmed that ex vivo flap transfection provides an efficient and localized viral transfection capability.

This study has shown that high levels of cellular transduction can be obtained by ex vivo adenoviral infusion into a composite tissue flap. Overall, the only apparent contraindication to vascular patency was infusion at supra-normal pressure levels. Viral transfection did not appear to be significantly changed when infusion was performed under cold versus warm ischemia. Although the viral perfusion conditions were varied slightly, there were consistent levels of transfection throughout the entire composite flap including the microvascular bed and surrounding tissue using either method. In addition, there was no evidence of systemic transfection in any experiment.

### 7. EXAMPLE 2

### Transfection of Microvascular Free Flaps with a Nucleic Acid to Ameliorate the Effects Diabetes Mellitus

7.1. Introduction

In this example, a rat superficial epigastric (SE) flap is genetically modified ex vivo with a nucleic acid encoding a therapeutic molecule of interest, i.e., the nucleic acid encoding proinsulin. The flap is re-implanted into the donor, where it functions as a neo-organ that delivers insulin following re-anastomosis.

The rat superficial epigastric flap is used as a model, principally because of its reproducibility and technical ease (Perry et al., 1984, Plast. and Recon. Surg. 74(3): 410-3). As will be understood by those skilled in the art, animal models (e.g., the mouse flap model (Cooley et al., 1998, Microsurgery 18(5): 320-3,), can be used to demonstrate the advantageous utility of the microvascular free flap method of the invention to deliver insulin systemically to an animal in need thereof The mouse model is advantageous because of the many well-defined models and transgenic animals available for it.

7.2. Materials and Methods

A replication-deficient adenovirus containing the CMV promoter driving expression of a proinsulin gene coding region is employed. A replication-deficient adenovirus containing the CMV promoter driving expression of a reporter, the β-galactosidase (lacZ) gene coding region (AdCMVlacZ), is used a control. A continuous microperfusion system is used to transfect the flap (Milas et al., Clinical Cancer Research. 3(12-1): 2197-2203, Dec 1997) as described above in Section 5.1.

Two control groups are also used in addition to the groups perfused with the proinsulin or lacZ vectors. One control group has flaps perfused with wild type adenovirus (to delineate flap toxicity from viral infection) and the other control group has flaps perfused with University of Wisconsin (UW) media alone (to delineate flap toxicity from the ex vivo perfusion protocol).

Following perfusion, the flaps are re-inset into the ipsilateral femoral vessels.

Animals are sacrificed at 1, 7 and 15 days and the flap, recipient bed, contra-lateral (unoperated) recipient bed, heart, liver, thymus and bran preserved in glutaraldehyde. Sections of tissues in the lacZ control group are stained for X-gal according to routine methods, the presence of which indicates successful transfer of the nucleic acid encoding proinsulin to the stained tissues. Polymerase chain reaction (PCR) is used according to standard methods to confirm the presence of the transferred coding sequences (i.e., proinsulin, β-galactosidase). Histologic specimens are also examined for cellular toxicity using haematoxylin and eosin (H and E) staining.

A further set of experiments is used to confirm that the microvasculature of the flap can be genetically modified to secrete physiologically significant levels of an encoded protein of interest, i.e., proinsulin. The well-established rodent diabetes mellitus model induced by streptozotocin (STZ) (Kolodka et al., 1995, Proc. Natl. Acad. Sci. USA. 92(8): 3293-7) is used. In these experiments, the therapeutic nucleic acid of interest exhibits sustained expression in the flap, a property that is not possible using adenoviral vectors. Thus, a liposomal transfection (lipofectin) or transfection with an adeno-associated (AAV) virus vector is used. Both methods appear to produce prolonged (and possibly permanent) gene expression as discussed above in Section 5.7.

Using previously described techniques (Spragg et al., 1997, Proc. Natl. Acad. Sci. USA. 94:8795-8800), a nucleic acid encoding rat proinsulin is packaged in cationic liposomes and is used to transfect harvested rat epigastric flaps. Control animals receive microvascular flaps transfected with liposomal β-galactosidase nucleic acid (lacZ) or perfused media. Fourteen days later, all animals are treated with STZ, inducing an iatrogenic form of diabetes mellitus. Treated and control animals are compared with respect to serum glucose levels, serum insulin levels, urinary ketones, change in body weight and survival over the ensuing six weeks. When a physiologic effect is demonstrated, a dose response curve is developed by serially excising portions of the flap and observing changes in the systemic glucose levels according to methods well known in the art.

At eight weeks, the modified flaps are excised and screened for clinical deterioration. At ten weeks, all animals are sacrificed. Western blot analysis is performed on the excised flaps to determine the presence of rat insulin in the excised flap tissue. This is compared with excised pancreatic tissue (which should be negative in STZ-treated animals) and with representative samples from other organ systems (i.e., heart, lung, liver, brain, etc.).

Experiments can also be performed in an animal, preferably a vertebrate animal, and more preferably a mammalian species such as mouse, rat, rabbit, guinea pig, dog, cat, pig, sheep, cow, horse, monkey, etc., to confirm that free genetic material can be effectively removed from a flap following ex vivo transfection and that no untargeted transfection occurs in the recipient animal following re-anastomosis.

7.3. Discussion

The methods exemplified above for the production of a systemically active flap have immediate applicability throughout medicine. The use of genetically modified free flaps as synthetic devices or pumps is desirable because it side-steps one of the major problems facing in vivo gene therapy, the balance between transfection efficiency and potential toxicity. Much of the prior gene therapy research as focused on the replacement of a single, soluble gene product such as insulin, factor VIII or erythropoietin. It has proven difficult to transfer enough genetic material in vivo to produce a clinical effect without risking serious toxicity.

Ex vivo modification circumvents this difficulty by removing the dangerous portion of gene therapy, i.e. transfection, from the recipient's physiology. There can be no hepatic, renal or respiratory toxicity from the transfection vector if the liver, kidneys and lungs are never exposed to it. Moreover, since these flaps are expendable, if problems develop the flap can be explanted or removed, a reversibility that is unattainable with systemic (in vivo) gene therapy.

### 8. EXAMPLE 3

### Transfection of Epigastric Free Flaps with IL-12

8.1. Introduction

In this example, a rat tumor model is used to confirm that a genetically modified pigastric free flap can act as a delivery vehicle for localized gene therapy with emonstrable antitumor effect. A rat subcutaneous tumor model is employed and the rat istiocytoma cell line AK-5 is used as the tumor source (Nandakumar et al., 1997, Cytokines Cell. Mol. Ther. 3(4): 225-32). Prior studies have demonstrated that this tumor is exquisitely sensitive to local IL-12 therapy when delivered via injection. The methods exemplified herein may also be applied to a mouse flap model (Cooley et al., 1998, Microsurgery 18(5): 320-3). As will be understood by those skilled in the art, animal models (e.g., the mouse flap model), can be used to demonstrate the advantageous utility of the microvascular free flap of the invention to act as a delivery vehicle for localized gene therapy. The mouse model is advantageous because of the many well-defined models and transgenic animals available for it.

8.2. Materials and Methods

AK-5 tumor implants (1 gm) are surgically engrafted into both groins of each experimental rat. Two weeks following engraftment, unilateral epigastric flaps are harvested and transfected with adenovirus containing a nucleic acid encoding murine IL-12 driven by the CMV promoter (AdCMVmIL-12). The flaps are then re-inset unilaterally over one tumor implant. The contralateral implant is left undisturbed and functions as an internal control for systemic L-12 antitumor effect. An additional external control group of animals is included, which consists of animals whose flaps are treated with wild type adenovirus.

After four weeks, all animals are sacrificed and the size of the AK-5 tumor implants compared between groups. Confirmation of adenoviral transfection is obtained by PCR analysis. The external and internal controls permit detection of a local IL-12 antitumor effect, attributable to the transfected epigastric flap.

### 9. EXAMPLE 4

### Transfection of Quadriceps Femoris Free Flaps with Endostatin or IL-12

This example exemplifies the transfection of a quadriceps femoris free flap in a rat head and neck tumor model with endostatin or IL-12.

9.1. Introduction

Endostatin has been shown to inhibit angiogenesis and cell migration, to prevent tumor growth and invasion. Endostatin is a circulating C-terminal fragment of collagen XVIII; it has been described to exhibit anti-angiogenic and tumor-regressing abilities with the unique property of a lack of acquired tumor resistance (O'Reilly et al., 1997, Cell 88: 277-285; Boehm et al., 1997, Nature 90: 404-407). It has been tested in both human clinical trials and likewise in preclinical models of gene therapy (Boehm et al., 1997, Nature 90: 404-407; Blezinger et al., 1999, Nature Biotech. 17:343-348; Feldman et al., 2000, Cancer Res. 60: 1503-06; Sauter, 2000, Proc. Natl. Acad. Sci. USA 97: 4802-07.

IL-12 has been shown to enhance killing of tumor cells by lymphocytes (Nastala et al., 1994, J. Immunol. 153: 1697-1706; Brunda et al., 1993, J. Exp. Med. 178: 1223-1230). IL-12 has also been previously evaluated for gene therapy in head and neck tumor models (Li et al., 2001, Arch. Otolaryngol. Head Neck Surg. 127: 1319-1324.

These two therapeutic molecules are employed because each has been used in clinical trials against other types of human malignancies and shown to be useful in therapy for head and neck cancers. Since many head and neck cancer patients with advanced disease undergo extirpations followed by reconstruction of the surgical defect with a microvascular free flap, they are ideal candidates for the protocol exemplified in this example, because as it can reduce residual tumor burden, local metastases and loco-regional recurrences.

Two different rat tumor models are used. First, a nude rat tumor model is used that develops subcutaneous xenografted human head and neck squamous cell carcinoma (SCC). Human head and neck SCC tumors account for 6% of all new cancers in this country and for 12,500 deaths each year (Landis et al., 1998, Cancer J. Clin., 1:6-29). A Wistar rat tumor model is employed that develops subcutaneous (rat) histiocytoma tumors.

9.2. Materials and Methods

Rat strains. Two different rat tumor models are used. A nude rat strain (Taconics) is used to determine the ability of a free flap transfected with adeno-associated viral vector (AAV) to deliver the anti-angiogenic protein endostatin to a local tumor site, and to ameliorate the growth and invasion of an established human squamous cell cancer (SCC) line. The human head and neck SCC line is used (SCC-1483, originally isolated from a SSC harvested from the retromolar trigone in a 54 year-old patient; provided by Dr. Peter Sacks, New York University Medical Center, see Sacks et al., 1988, Cancer Res., 48: 2858-2866). Unlike the mouse, which has an abundance of available head and neck SSC lines available for study, no head and neck SSC line is available that is derived from a rat.

A nude rat is used because it does not immunologically reject a xenografted human SSC line. This minimizes the effects that the host immune response has against the tumor. It has been shown, in addition, that endostatin does not interfere with the vascularization necessary for normal wound healing (Berger et al., 2000, J. Surg. Res. 91: 26-31), and therefore no problems with healing of-the transfected free flap should be encountered. The nude does not have competent T-cells and therefore, this strain cannot be used to study the effects of an immunostimulatory molecule such as L-12, which exerts it biologic effects through the stimulation of host lymphocytes.

A second rat model, the Wistar rat strain (Taconics), is used to examine the effects of an AAV delivering the immunostimulatory cytokine IL-12 on growth and invasion of a syngeneic rat histiocytoma cell line, AK-5, which is known to be sensitive to the effects of IL-12 (Jyothi et al., 2000, Cancer Immunol. Immunother. 49: 563-572; Nandakumar et al., 1997, Cytokines Cell Mol. Ther. 3:225-232). AK-5 cell line was originally isolated from a Wistar rat strain, and therefore an immunocompetent syngeneic Wistar rat can be utilized for the experiments that utilize the AAV-IL-12 constructs. Unlike the case with endostatin, the use of an immunocompetent rat is preferable, as IL-12 achieves its anti-tumor effects by mobilizing and stimulating the host immune response. Furthermore, although this particular histiocytoma was not originally isolated from the head or neck area, histiocytomas are certainly a subset of head and neck malignancies, and this cell type may therefore be employed.

The rats used are 6-8 months old. To ease the technical difficulties of performing a microvascular anastamosis in a rodent, rats are used that weigh approximately 300 grams. Preferably only males are used in these experiments. Approximately 50 animals are utilized.

Tumor cells. The tumor cells are grown in standard tissue culture conditions in humidified incubators with a 21% oxygen, 5% carbon dioxide atmosphere at 37° C. The SSC-1483 cells are adherent cells that grow as a monolayer, while the AK-5 cells. grow in suspension. Prior to implantation in animals, both types of tumor cells, SSC and histiocytoma, are stably transfected with a plasmid containing a nucleic acid encoding beta human chorionic gonadotropin (beta-hCG) in order to allow noninvasive measurement of tumor burden by measuring beta-hCG in the urine of the animals.

The expression of the beta-hCG nucleic acid is under control of the CMV promoter, which produces high levels of expression in mammalian cells. The plasmid is transfected using a cationic liposomal method (GeneJammer Transfection Reagent, Stratagene), and grown in the presence of neomycin for 3-4 weeks in order to select clones that have stably integrated the beta-hCG plasmid. Expression of the beta-hCG plasmid is verified by Western blotting; those clones that express the highest level of beta-hCG are expanded and used for the animal experiments. From this point on, the AK-S cells and the SCC-1483 cells that are used are stably expressed and secrete beta-hCG.

The rationale for transfecting these cells with beta-hCG is that it permits the accurate measurement of the growth of tumors and their response to gene therapy with IL-12 or endostatin in a non-invasive manner. It has been demonstrated that the expression of beta-hCG in a similar system correlates directly with tumor burden (Shih et al., 2000, Nat. Med. 6: 711-714). Since this is a human protein, and since all rats used are preferably male, any beta-hCG secreted into the urine are derived from the tumor cells.

Tumor cells are injected percutaneously into a subdermal pocket in one thigh of each experimental animal. A total of 5×10⁶ cells can injected into one thigh of each rat. Experiments using AK-5 cells have shown that this number of cells reliably results in a 1×1 cm³ tumor nodule that is easily palpable 2 weeks following the surgery. The same number of SCC-1483 is implanted; however, this number may be adjusted to reliably produce a sizable tumor,

Tumor mass is measured non-invasively by measuring the amount of beta-hCG in the urine (such measurement services may be obtained commercially, from e.g., New York University Clinical Laboratories). A beta-hCG urine sample is measured by placing each rat in a metabolic cage to collect its urine over 2 hours. 100 microliters of each specimen is measured. A baseline value is determined the day prior to surgery, and corresponds to tumor burden prior to surgical and gene therapy interventions.

Construction of adeno-associated viral vectors (AAV). The AAV vectors are constructed in a helper virus-free system that is commercially available (Stratagene). The benefit of using this system is that it allows the production of AAV-2 vectors without the need for live helper viruses, which are traditionally necessary for AAV production, thereby greatly adding to convenience, purity and safety. Briefly, basic molecular cloning techniques commonly known in the art are used to place an insert containing the coding sequence of either the human IL-12 gene or the murine endostatin gene, into the multiple cloning site of a shuttle vector (pCMV-MCS). This shuttle vector places the CMV promoter upstream of the inserted nucleic acid. The entire cassette, consisting of the upstream CMV promoter and the nucleic acid of interest, is then removed from the shuttle vector via restriction enzyme digestion and ligated to a pre-linearized pAAV vector. This vector contains the above cassette flanked by two inverted terminal repeats (ITRs) necessary for virus replication and packaging.

The pAAV plasmid vector is then transfected into a HEK293 packaging cell line along with two helper plasmids that supply the rest of the AAV components necessary to produce viable, infectious virions (namely, the rep and cap genes, as well as two envelope proteins, E2A and E4). The HEK293 cell line provides the E1 envelope protein, and produces a high titer of recombinant AAV.

Once constructed and purified by ultracentrifugation, the AAV is titered. This is done by adding serial dilutions of the AAV stocks to a permissive cell line (HT1080, ATCC #CCL-121) and performing a plaque forming assay. Typical titers obtained range from 10⁶ to 10⁹ plaque forming units per mL (PFU/mL). Once titered, the AAV stocks are aliquoted and stored at −80 degrees until used in the animal experiments.

Transfection of quadriceps femoris free flap. The experimental rats are anesthetized and the thigh containing the tumor nodule is shaved and washed with alcohol followed by a betadine paint. A longitudinal incision is made down through the skin to expose the quadriceps femoris muscle; care is taken to identify and not disturb the tumor nodule. The muscle is dissected circumferentially to free all attachments, and the femoral vein and artery are identified and dissected to their origin in the groin. The edges of the muscle are then cauterized to prevent egress on the virus from the wounded edges later during flap perfusion.

Clamps are then maintained on the proximal femoral vessels and the vessels are divided distal to the clamps. The flap is then removed from the vicinity of the anesthetized rat to prevent accidental contamination of the wound bed with AAV during the flap transfusion. A 0.2 mm inner diameter infusion catheter (BioTime Micro-cannula, BioTime, Inc., Berkeley, Calif.) is placed in the artery and secured with suture(s). The flap is then perfused, ex vivo, through the arterial catheter, with 20 cc of IX Dulbecco's PBS at a constant perfusion pressure to flush out blood from the flap vessels. Flap infusions are performed at physiologic perfusion pressures between 80-160 mmHg (±5 mmHg) to reduce barotrauma from the infusion itself. The infusion rate and pressure is monitored using a Harvard Apparatus pump (Holliston, Mass.) and pressure gauge. During this infusion, the efferent vein is clamped to prevent outflow of the solution and maximize transfection efficiency within the free flap tissues.

The flaps are transfused with 5 mL of a PBS solution containing AAV (either AAV-IL I2, AAV-Endo or AAV-lacZ); two different experimental concentrations (low and high) of each virus are used. In the low titer group, the AAV vectors are delivered at a concentration of 5×10⁶ PFU/mL. The high titer group's concentration is 5×10⁹ PFU/mL.

The solution is allowed to dwell in the flap for one hour. We have previously shown that this protocol maximizes viral infectivity while minimizing flap ischemia. Flap survival is not compromised by this ischemic time.

After infusion, the venous occlusion clamp is removed and the flap is flushed again with 20 ml of PBS. All flap surfaces are rinsed to wash out any free virus. The vessels are then re-anastomosed to the proximal femoral vessels using 9-0 nylon sutures under an operating microscope. The flap is re-inset into the groin directly adjacent to the previously identified tumor and the incisions closed with interrupted nylon sutures. The animals are then placed in isolation cages in a Biosafety Level 2 room.

Data acquisition. Following transfection of the flaps, the urinary beta-hCG levels of the animals are measured every other day. The level of beta-hCG in the urine is plotted as a function of time in each animal. The levels are determined by a beta-hCG ELISA. One half of the animals are sacrificed at four weeks following surgery and one half are sacrificed at eight weeks following surgery. The residual tumor is identified, dissected free and weighed. The tumor is then fixed overnight in 10% formalin and processed for histologic study using routine methods.

Some paraffin sections are stained with a CD3 1 antibody (Clone Santa Cruz Biotechnology) as a way to gauge the number of blood vessels present in the tumors and correlate the number of blood vessels with the therapy used. In addition, apoptotic index is measured with a TUNEL assay (Roche) to screen for programmed cell death (as opposed to necrosis, which is nonspecific to therapy).

To confirm biochemical presence of the transgene (encoding IL-12 or endostatin) at the time of animal sacrifice, a sample of the transfected muscle flap is homogenized, total presence of the lacZ control transgene is conveniently assayed for in a few animals by colorimetric staining for P-galactosidase activity using routine methods well known in the art.

In certain cases in which a response to tumor therapy is seen earlier than four weeks or later than eight weeks, the endpoints of the experiment may be adjusted accordingly. Statistical analysis. Two types of data are collected. The first type of data are the absolute values of the beta-hCG secreted into the mouse urine. The data are preferably gathered using methods such as used by a commercial clinical laboratory (e.g., the New York University Medical Center clinical laboratory), so that values are obtained that can be compared among specimens from different animals and gathered at different time points, even if the assays are not run simultaneously. Values of hCG in the urine are determined at different time points (before treatment and after treatment). A Mann-Whitney analysis may be used, as needed, to determine the statistical significance between these different groups at different time points. The same analysis is performed when comparing the dry weights of the tumors following sacrifice at the designated end points (four and eight weeks post-transfection).

The data obtained from immunohistochemistry (degree of apoptosis and vessel counts) are treated as qualitative, and the degree of apoptosis and of vascularity in the tumor specimens is graded in a relative manner.

Animal husbandry. All animals are housed according to standard institutional and federal guidelines. They are placed in standard cages, 12-hour light cycles and fed standard rodent chow. After the animals undergo surgery they are transferred to a NIH Biosafety Level 2 room where they remain until they are sacrificed. In addition, nude rats are placed in a pathogen-free room. For measurement of urinary beta-hCG levels, the animals are placed in a metabolic cage for 2 hours every other day. The urine is collected and stored frozen until beta-hCG levels are assayed. Animals are closely monitored, particularly after surgery. Pain control is given for 3 days following surgery with IM buprenorphine. Any animals that develop infections, sepsis, wound breakdown or flap necrosis are euthanized. Euthanasia is preferably done by carbon dioxide narcosis. This method is consistent with the recommendations of the Panel of Euthanasia of the American Veterinary Medical Association.

9.3. Discussion

A free flap procedure being performed by a surgeon for reconstructive purposes can also serve a second therapeutic function by releasing an anti-tumor agent. This form of local gene therapy has immediate clinical applications in those situations where obtaining local control of an oncologic process is difficult or impossible (i.e., unresectable tumors). Areas in which the methods exemplified herein can prove useful would include head and neck tumors, soft tissue sarcomas, central nervous system malignancies, etc. In addition, recent evidence suggests that the metastatic potential of residual cells in a tumor bed can be suppressed by local immune therapy (Fidler, 1999, Can. Chemo. And Pharm. 43 Suppl: 83-10, 1999). Thus this approach may be used situations where local control is less problematic, such as breast cancer. As such, the methods exemplified herein have wide ranging applicability to many forms of oncologic reconstruction.

### 10. EXAMPLE 5

### Expansion of Microvasculature of Microvascular Free Flaps

In this example, a microvascular free flap is maintained long-term in a bioreactor and the conditions suitable for angiogenesis are analyzed. The microvasculature of the free flap, including blood vessels such as capillaries, venules, arterioles, veins, arteries, etc., is expanded ex vivo using the conditions that are determined to be suitable for angiogenesis. The free flap with expanded vasculature can then be reimplanted into a recipient, where its survival is enhanced and/or its ability to deliver a product of interest improved, owing to its expanded vasculature. The free flap with expanded vasculature can also be used to provide a microvascular framework around which differentiated and stem cells can proliferate, differentiate and integrate into resident tissue.

10.1. Introduction

Hypoxia is the primary in vivo regulator of angiogenesis occurring synchronously with tissue growth during embryologic development (Semenza et al., 1999, Ann. NY Acad.Sci. 874: 262-8), placental development (Folkman et al., 1971, N. Engl. J. Med. 285: 1182-6), wound healing (Knighton et al., 1981, Surgery 90: 262-70), and tumor growth (Folkman et al., 1971, N. Engl. J. Med. 285: 1182-6). HIF-1α is a transcription factor whose expression is rapidly induced by hypoxia (Jiang et al., 1996, Am. J. Physiol. 271:C1172-80). It binds to hypoxia response elements thereby promoting the transcription of several proangiogenic genes including VEGF (Semenza et al., 1998, Curr. Opin. Genet. Dev. 8: 588-94). HIF-1α expression is low in normoxia, but it increases exponentially as oxygen concentrations decrease below 5%, with maximal changes occurring between oxygen concentrations of 0.5-2% (values representative of ischemic tissue) (Jiang et al., 1996, Am. J. Physiol. 271:C1172-80). Thus, 1% oxygen is used to reproduce the in vivo induction of angiogenesis within the system.

While hypoxia generally induces angiogenesis in harvested microvascular beds, it certain embodiments, angiogenesis may be stimulated under normoxic conditions. This can be achieved by supplementing the perfusing vascular media with hypoxia-inducible, proangiogenic growth factors such as VEGF (Shweiki et al., 1992, Nature 359: 843-5; Brogi et al., 1996, J. Clin. Invest. 97: 469-76; Yamagishi et al., 1999, Lab. Invest. 79: 501-9). Transfection of the vasculature with VEGF has been shown to increase vascular density in animal models of ischemia (Takeshita et al., 1994, J. Clin. Invest. 93: 662-70; Takeshita et al., 1996, Lab. Invest. 75: 487-501). A VEGF-encoding adenovirus is used to transfect the microvasculature of tissue beds to induce angiogenesis in normoxia, and augment angiogenesis in hypoxia.

10.2. Materials and Methods

Sprague-Dawley rats are used as experimental subjects. The rat quadriceps femoris flap (Dogan et al., 1999, J. Reconstr. Microsurg. 15: 433-437) is used as the source of tissue. This particular tissue has high vascular density, its low metabolic demand as well as its amenability to harvest. The microvascular free flap is removed under sterile conditions from the leg of the rat. The main vessels have an inner diameter of approximately 1.5 mm, and can be routinely cannulated and re-anastamosed using microvascular techniques. Following harvest, the blood remaining in the microvasculature is drained by perfusion with a heparin-solution through the artery. Any leaks from the cut edges of the tissue are sealed with a coagulator, leaving the vein as the sole means of fluid egress.

The tissue is then inserted into the sterile environment of a bioreactor, where the artery and vein are both cannulated and thereby connected to the bioreactor using methods commonly known in the art (FIGS. 3 and 4). At this point, the tissue, which contains muscle and most importantly, the microcirculation, is ready to be perfused and utilized in the experiments described below.

First, the oxygen tension of the bathing solution is varied to activate hypoxia-driven mechanisms of angiogenesis. Specific endpoints that are determined include activation of hypoxia inducible factor-1α (HIF-1α), VEGF production and histological determination of blood vessel density. The tissue bed is also transfected with a VEGF-encoding adenovirus to enhance angiogenesis by a different mechanism. The tissue beds used in this example can be used to provide a microvascular framework around which differentiated and stem cells can proliferate, differentiate and integrate into resident tissue.

Hypoxic culture conditions. To test the efficacy of hypoxia as stimulus for angiogenesis, harvested tissue beds are exposed to oxygen tensions of 20%, 10% 5% and 1%, which are representative of normal physiologic oxygen tensions in the atmosphere, arterial blood, normoxic tissue and hypoxic tissue respectively. HIF-1α function is expected to be isolated to the 1% oxygen group, in which significant angiogenesis should be observed. Each group consists of tissue bed exposure to hypoxia for periods of 0, 1, 3 and 7 days (n=5 for each group).

VEGF adenoviral transfection of flap or vasculature. VEGF is used as a cell marker and to test for the functionality of the transformed tissue. An adenovirus encoding human VEGF₁₆₅ is used (source: Dr. Ronald Crystal, Cornell Medical College). The adenovirus is grown in a 293 cell line (ATCC), purified in a cesium chloride gradient by ultra centrifugation, and titered. The adenoviral construct is confirmed by Western blotting with a VEGF antibody (Santa Cruz Biotechnology). 1×10⁹ PFU/mL is delivered via the intravascular route; this transfects most of the endothelial cells.

Tissue analysis. In order to confirm that the tissue bed is adequately responding to its hypoxic environment, the level of HIF-1α protein is determined by immunoassay. Following exposure to hypoxic conditions, a 500 mg piece of tissue is removed, and homogenized on ice with a nuclear extraction kit (ActiveMotif) in the presence of protease inhibitors (Complete Mini-tablet, Boehringer Mannheim). The nuclear extracts are quantified (BCA-200 Protein Quantification kit, Pierce), aliquoted, and frozen at −80 degrees until ready for use.

50 ug of nuclear extract is placed in each well of a 7.5% SDS-PAGE gel and separated. The proteins are transferred to a PVDF membrane (Immobilon). The membrane is reversibly stained with Ponceau S stain to verify even transfer. The membrane is then be washed, blocked for 1 hour with SuperBlock buffer (Pierce) and then incubated overnight with a 1:200 dilution of a monoclonal antibody, which recognizes rat HIF-1α (Clone Hia67, Novus Biologicals) at 4° C. The next day the membrane is washed with TBS/0.05% Tween-20, and incubated with an HRP-conjugated anti-mouse secondary antibody (Amersham).

The blot is then exposed to enhanced chemiluminescence substrate (ECL Plus, Amersham) and exposed to radiographic film. The signal is then developed; bands are scanned and signal intensity is quantified with densitometry software (Kodak Image Analysis Suite).

VEGF Northern. As an initial marker of tissue response to hypoxia, VEGF rnRNA is measured in control and experimental groups at 0, 6, 12, 24 and 72 hours. At the end of each time point, tissue beds are homogenized in Trizol Reagent (Life Technologies, Inc.) and purified according to the manufacturer's instructions. Amount and purity of RNA are determined by spectrophotometry. Northern blot analysis are carried out by separating 10 μg of total RNA via electrophoresis in a denaturing 1% agarose gel. After electrophoresis, the RNA is transferred to a nylon membrane (Ambion) by vacuum transfer (Bio-Rad Vacutransfer system) and UV-crosslinked to the membrane (Stratalinker). The membrane is then be probed with ³²P-labelled cDNA probes for VEGF (the 165 and 180 bp isoforms) and 18S RNA (generous gift of Dr. Jeffrey Isner, St. Elizabeth's Medical Center). Blots are exposed to film and analyzed using computer densitometry software.

VEGF ELISA. As another marker of tissue response to hypoxia, VEGF protein production by the microvascular beds is measured. This is done by performing an ELISA analysis (Boehringer Mannheim) of the perfusion solution and bathing solution after 1, 6, 12, 24, and 72 hours of tissue bed exposure to hypoxia.

Tritiated thymidine proliferation assay. Hypoxia induces angiogenesis. Subsequently, in order to obtain a measure of global proliferation within the tissue and vascular bed, tritiated thymidine incorporation assays are performed to quantify cellular proliferation within the perfused tissue bed. 200 μCi of tritiated thymidine is added to the perfusion solution and allowed to circulate for 3, 6, 12, and 24 hours. After being thoroughly flushed, the rate of thymidine incorporation within a sample of tissue is deterriined by homogenizing the tissue and measuring the degree of radioactivity of TCA-precipitated material in a liquid scintillation counter.

Histological analysis. At least three histological techniques may be employed to accurately quantify changes in blood vessel density within the tissue beds. First, 500 kg of FITC-labeled Bandeiraea simplicifolia lectin I (Vector Laboratories) is infused into the perfusion media for 30 mm prior to tissue harvest to label the microvasculature (Thurston et 5 al., 1998, Am. J. Pathol. 153: 1099-112; Thurston et al., 1999, Science 286: 2511-4). The tissue is then harvested and fixed in 4% paraformaldehyde for 1 hr, washed with PBS, and snap-frozen in liquid nitrogen. Tissue is sectioned at 40 μm cuts and 10× high power fields analyzed under fluorescent microscopy to quantify vascular density.

Immunohistochemical staining is also performed on tissue fixed in 10% formalin and embedded in paraffin. Endothelial cells are stained with a polyclonal antibody against rat CD3I (DAKO); the secondary antibody is linked to horseradish peroxidase, and a colorimetric substrate such as DAB may be used. Capillary density is again determined by histological examination of 10 randomly selected fields from central and peripheral segments of the tissue.

To histologically identify areas of neovascularization, a subset of tissues is analyzed for cellular proliferation using a BrdU incorporation assay (BD Pharmingen). BrdU (1 mg/ml) is infused through the perfusion solution for 3, 12, 24, and 72 hours prior to tissue harvest. Tissue is fixed, e.g., with formalin fixation, embedded in a histological medium such as paraffin, and stained using a mouse monoclonal antibody to BrdU (1:200; DAKO) followed by an FITC-conjugated goat anti-mouse IgG (1:200 dilution; Jackson ImmunoResearch).

To specifically identify proliferating endothelial cells from all other cell types, sections are double-stained with the CD31 antibody discussed above, which is followed by detection with a rhodamine-labeled secondary antibody against rabbit IgG. The sections are then measured under a dual pass-band filter using a fluorescent microscope (Olympus BX-51).

Under certain conditions, the hypoxic conditions designed to enhance vascularization may lead to cell death if continued for a chronic period. In these cases, the hypoxic environment may be maintained for a shorter time period.

### 11. EXAMPLE 6

### Introduction and Long-Term Maintenance of Bone-Marrow Derived Stem Cells into a Microvascular Free Flap

11.1. Introduction

This example exemplifies the introduction into and long-term maintenance of bone-marrow derived stem cells in a microvascular free flap.

It is known in the art that bone marrow-derived stem cells can contribute to vascular tissue (Luttun et al., 2002, Vascular progenitors: from biology to treatment, Trends Cardiovasc. Med. 12(2):88-96; Gunsilius, Bone marrow-derived endothelial cells for therapeutic angiogenesis and antiangiogenesis: facts and visions, 2002, J. Hematother. Stem Cell Res. 11(1): 153-5, Isner et al., 2001, Bone marrow as a source of endothelial cells for natural and iatrogenic vascular repair, Ann. NY Acad. Sci. 953:75-84). Bone marrow-derived stem cells can be transplanted, e.g., from a Tie2/lacZ transgenic mouse to repopulate the marrow of an irradiated, syngeneic host. Two assays of in vivo neovascularization well known in the art, an in vivo MATRIGEL™ assay (see, e.g., Eliopoulos et al., 2002, Gene Ther. 9(7):452-62) and a corneal micropocket assay (see, e.g., Parry et al., 1999, Nucleic Acids Res.27(13):2569-77; Shin et al., 2000, Cornea 19(2):212-7) can be used to demonstrate that bone marrow-derived endothelial progenitor cells (EPCs) account for up to 26% of newly formed endothelial cells (ECs) in these assays. Adhesion of bone marrow-derived stem cells (i.e., EPCs) to endothelial beds in vitro is also known to be enhanced by pro-inflammatory cytokines such as TNFA. In the present example, bone-marrow derived stem cells are introduced into a microvascular free flap in bioreactor and maintained long-term. Using this approach, the microvasculature of a free flap may be expanded ex vivo by introducing EPCs that differentiate into endothelial cells that contribute to the expanded microvasculature.

11.2. Materials and Methods

Animals. Anatomically identical microvascular beds from athymic nude rats (Hsd:RH-rnu; Harlan) are used. Bone marrow donor cells are obtained from transgenic mice ubiquitously expressing green fluorescence protein or lacZ (FVB/NJ-TgN(GFPU)SNagy and B6. I 2957-Gtrosa26, respectively).

Bone marrow cell isolation. Bone marrow cells are harvested from male transgenic mice by flushing the tibias and femurs with PBS+5 μM EDTA. To purify a mononuclear cell (MNC) population, bone marrow cells are filtered (30 cm), centrifuged with Histopaque 1083 (Sigma), and exposed to ammonium chloride for red blood cell lysis. Approximately 1×10⁷ bone marrow-MNCs are harvested from eacittransgemc mouse.

A portion of bone marrow-MNCs are then used to generate populations of HSCs and MSCs. Lin⁻ ckit^{POS}Sca-1⁺ cells are isolated from the bone marrow-MNCs as previously described (these are the HSC population) (Orlic et al., 2001, Proc. Natl. Acad. Sci. USA 98(18):10344-49; Orlic et al., 2001, Nature 410:701-5). MSCs are cultured based on techniques well known in the art that exploit their adherence properties (Pittenger et al., 1999, Science 284: 143-7). This results in three cell populations that are used for study: (1) freshly isolated bone marrow-MNCs, (2) MSCs, and (3) HSCs.

Bone marrow cells from transgenic mice are functional in the tissues of nude rats, and allow for the successful restoration of hematopoiesis by transplanting mouse bone marrow cells to lethally irradiated nude rats (Kawamoto et al., 2001, Circulation 103: 634-7). Mouse bone marrow cells can be determined to be functionally equivalent to bone marrow cells from homologous rats within a bioreactor system by performing identical experiments with fluorescent-labeled (DiI; Molecular Probes) bone marrow cells from nude rats. DiI is used for cell tracking because it offers long-term detection (as much as 28 days) (Spotl et al., 1995, Cytometry 21: 160-9; Kalka et al., 2000, Proc. Natl. Acad. Sci. USA. 97: 3422-7).

Cells in suspension are marked at a concentration of 2.5 μg/ml in PBS for 5 mm at 37° C. and 15 mm at 4° C. In addition, all microvascular tissue beds are obtained from females rats, thus allowing for gender mismatch detection of injected cells by FISH analysis for the Y chromosome (Weier et al., 1994, Genomics 21: 641-4).

Primary hepatocyte isolation. Although differentiated cells are limited by their rapid de-differentiation and short-term growth in culture, co-culture with other cell types and the presence of a three-dimensional scaffold improves their growth in culture (Zimmermann et al., 2002, Circ. Res. 90: 223-30; Bhandani et al., 2001, Tissue Eng. 7: 345-57). The survival and fate of mature cells in the bioreactor system may be determined as follows. Primary hepatocytes are isolated from nude rats according to a two-step collagenase perfusion method (Reese et al., 1981, In Vitro 17: 93540). Prior to removal of the liver, the portal vein is cannulated and flushed with saline. The liver is then be surgically removed and flushed with 0.65 mg/ml of collagenase type IV (Sigma).

The cell suspension is filtered and washed by centrifugation, and cell viability is assessed by trypan blue; generally only cultures achieving viability over 85% are used for the study. Cells are fluorescently labeled with Dil as described above. The media may be supplemented with substances that have been shown to limit primary hepatocyte de-differentiation and promote their growth (e.g., DMSO+ copper, iron, zinc) (Cable et al., 1997, Hepatology 26: 1444-57).

Delivery of cells. To deliver bone marrow cells through an intra-vascular approach, bone marrow-MNCs and MSCs (2×10⁵, 1×10⁶, 5×10⁶) are suspended in the perfusion solution. Because far fewer HSCs can be isolated from the bone marrow, 2×10³·1×10⁴, and 5×10⁴ cells are delivered in the same manner. The tissue are then perfused at intervals ranging from 1 min to 1, 6, 12, 24, and 72 hr.

To test the ability of cytokines to enhance the egression of bone marrow cells, the effects of pretreatment with factors known to enhance the expression of adhesion molecules on endothelial cells ([kuta et al., 1991, Immunology 73: 71-6; Mackay et al., 1993, J. Exp. Med. 177: 1277-86; Kukreti et al., 1997, Blood 89: 4104-11; Dejana et al., 1988, J. Clin. Invest. 82: 1466-70) may be monitored. TNF-A or IL-I are two well-known chemokines that enhance vascular adhesion by inducing endothelial cell expression of molecules such as VCAM-1, ICAM-1, E-selectin, and P-selectin (Dustin et al., 1986, J. Immunol. 137: 245-54; Hakkert et al., 1991, Blood 78: 2721-6; Pober et al., 1986, J. Immunol. 137: 1893-6; Hashimoto et al., 1994, Iflammation 18: 163-73; Myers et al., 1992, Am. J. Physiol. 263:-C767-72). Prior studies with circulating adult stem cells (i.e., endothelial progenitor cells) indicates that adhesion to mature endothelial cells is also stimulated by these cytokines. Therefore, in an attempt to maximize the incorporation of intravascular-delivered bone marrow-derived cells, the perfusion solution is supplemented with either TNF-α (1 ng/ml) or IL-1 (1 ng/ml) and the microvascular beds perfused for 6 hours. Following cytokine pre-treatment, either bone marrow-MNCs, HSCs, or MSCs are delivered as described above.

In certain experiments, bone marrow cells and differentiated hepatocytes are also delivered from an “outside-to-inside” approach by bathing microvascular tissue beds in cell-containing media. bone marrow-MNCs, HSCs, MSCs (at concentrations as above), and rat hepatocytes (1×10⁵, 1×10⁵, 5×10⁵) are suspended in growth media at concentrations similar to above, and the short- and long-term effects bathing these tissue beds in cell solutions are determined.

Tissue analysis. To analyze the ability of bone marrow cells to migrate into the microvascular free flap tissue, tissue is analyzed 1,3,6, and 12 hours after discontinuing cell infusion or suspension. A maximum time interval of 12 hours is used for egression studies because bone marrow stem cells are proliferative within 12 hours and longer time periods may reflect not only migration but stem cell proliferation and differentiation (Quesenbenry et al., 1998, Proc. Natl. Acad. Sci. USA 95: 15155-7; Nilsson et al., 1997, Blood 90: 4646-50).

To determine the viability and long-term fate of bone marrow cells in tissues, specimens at 1, 3, 7, and 14 days (or the maximum time point of survival that the system allows) are screened for the presence of these cells.

For the detection of lacZ expressing bone marrow cells, tissues are fixed in 1% paraformaldehyde (PFA) for 1 hr, washed with PBS, and stained overnight in X-gal staining solution in a dry incubator. Tissues are then embedded in paraffin and sectioned at 10 Fm. As another marker for lacZ expression, immunohistochemical staining can be performed using mouse monoclonal anti-β-gal antibody (Roche; clone D19-2F3-2) at a 1:1000 dilution.

To detect fluorescent-labeled bone marrow cells or hepatocytes (i.e. GFP, DiI), tissues are snap-frozen in liquid nitrogen and sectioned with a cryostat. As mentioned above, FISH analysis for the Y chromosome permits further distinction between administered cells and cells comprising the microvascular tissue bed.

Cell function and differentiation experiments. The experiments described above are used to establish the viability of various cell types in the microvascular bed. Since bone marrow, HSC, and MSC transplantation have all been shown to enhance and contribute to new blood vessel growth in ischemic tissues (Wang et al., 2001, J. Thorac. Cardiovasc. Surg. 122: 699-705; Gao et al., 2001, Cells Tissues Organs 169: 12-20; Kamihata et al., 2001, Circulation 104: 1046-52; Kocher et al., 2001, Nat. Med. 7: 430-6) the tissue is analyzed for evidence of bone marrow cell contribution to neovascularization by using the methods discussed above.

If substantial numbers of MSCs are present in the tissues, experiments are conducted to determine whether these cells can be directed to differentiate into phenotypes (osteogenic) similar to what has been described in other culture systems. For instance, MSCs have been shown to differentiate into an osteogenic phenotype in the presence of dexamethasone or myocytes in the presence of 5-azacytidine and amphotericin B (Wakitani et al., 1995, Muscle Nerve 18: 1417-26). For osteogenesis studies, osteogenic supplements (100 nm of dexamethasone, 0.05 mM ascorbic acid, and 10 mM β-glycerophosphate) are administered in the bathing fluid and/or perfusion solution (Jaiswal et al., 1997, J. Cell Biochem. 64: 295-312). These inductive conditions are applied from 1 week or longer.

Osteogenesis is assessed histologically for calcium deposits (staining with Alizarin Red 5) and a calcium assay kit (Sigma). In addition, tissue osteocalcin levels are determined via Northern blot analysis, as discussed above, using rat complementary DNA (cDNA) probes for osteocalcin (286 bp). Rat hepatocytes are characterized by measuring albumin levels in the perfusion solution and bathing fluid with an ELISA specific for rat albumin (R&D Systems).

Studies have shown that different mouse strains result in varying degrees of MSC culture purity (Phinney et al., 1999, J. Cell Biochem. 72: 570-85). For instance, bone marrow from FVB background mice produce reliable cultures, while mice of C56/BL6 and 129 background do not thrive as well. Therefore, in certain experiments, bone marrow cells from FVB wild-type mice may be transfected with lacZ.

The method of the invention for extra-vascular of delivery by cell suspension provides the capability for external manipulation as well as a more even distribution of cells. However, because adhesion is an important aspect of cellular growth, in certain cases only trace numbers of cells may incorporate into the tissue when kept in suspension. In such cases, cells will be delivered via suspension on a matrix (i.e., Matrigel or collagen). Previous studies with bone marrow stem cells suggest that both of these techniques are feasible and effective (Ferrari et al., 1998, Science 279: 1528-30; Eliopoulos et al., 2002, Gene Ther. 9(7):452-62; Al-Khaldi et al., 2002, Abstract Presentation, American Heart Association Scientific Session).

In other embodiments, the extra-vascular delivery experiments discussed above may be performed using a human hepatoma line (C3A) (Wang et al., 1998, Cell Transplantation 7:459-68).

### 12. EXAMPLE 7

### Implantation of a Microvascular Bed into a Recipient

In this example, three different kinds of microvascular free flap are returned to an in vivo environment in a host: i) an unaltered free flap, ii) an “expanded” free flap in which the microvasculature of the free flap has been expanded, and iii) a “cell-seeded” free flap.

12.1. Introduction

Because microvascular free flaps offer the advantage of an intact blood supply, re-integration is technically feasible as compared with other engineered tissues that rely on engraftment from surrounding tissue. Using the methods of the invention described hereinabove, a microvascular free flap is returned to an in vivo environment through vascular re-anastamosis. Analogous to organ transplantation, true success is not merely based on the technical placement of the organ, but rather durable physiologic function following re-implantation.

12.2. Materials and Methods

Tissues that are successfully maintained in the bioreactor system according to Section 5.1 above, are re-implanted into animals in order to examine vascular patency and tissue survival. Microvascular free flaps that have been modified (i.e., expanded according to the methods described hereinabove) or engrafted with introduced cells (according to the methods described hereinabove) are also re-implanted, to assess whether cellular differentiation either continues, reverts, or changes owing to new environmental factors. At 1 week or 2 weeks of maintenance in the bioreactor, tissue is removed from the bioreactor and anastamosed to a recipient animal. These time points represent the full spectrum of ex vivo viability determined in specific aims of earlier experiments. For these sets of experiments, gender mismatch is modified such that all donor tissues are of male origin (i.e., microvascular free flap ± administered cells) and the recipient animal are female nude rats. Again, nude rats are chosen in order to avoid transplant rejection.

Surgical anastomosis. The microvascular tissue free flap is connected to branches of the femoral vessels in an entopic location using an operating microscope. Standard surgical microsurgical techniques are employed, including placement of interrupted 9-0 and 10-0 nylon sutures to create a vascular anastamosis.

Assessment of viability. Most tissue analyses of the microvascular free flaps can be conducted in a manner identical to those conducted for their in vitro counterparts as described hereinabove. At various time points following implantation (1, 3, 7, 14, 28, and up to 56 days), the tissue is harvested and analyzed histologically. Tissue is stained and prepared as discussed above; lacZ expressing bone marrow cells are stained with X-gal solution and stained with anti-P-gal antibody and fluorescent-labeled cells (i.e. GFP, Dil) are identified in frozen sections.

As discussed above, FISH analysis for the Y chromosome can be used to further distinguish between donor and recipient cells, especially in transition zones. In addition to confirming viability, these staining techniques can also provide important data regarding the location of the cell engraftment and potential migration through the parenchyma.

The reinsertion of the microvascular free flap in a partial cutaneous fashion allows assessment of flap viability on a gross level through routine visual examination. Cell proliferation can be assessed through a BrdU assay, as commonly known in the art, e.g., as slightly modified from Section 10 above. Animals receive intraperitoneal injections of BrdU (1 mg/ml) 3 or 12 hrs prior to sacrifice, and tissue is stained with antibody to BrdU as described above.

As outlined in Section 11 above, functional characteristics of resident cells within the microvascular free flap are tested. Osteogenesis of bone marrow stem cells is examined by administering 100 nM dexamethasone (subcutaneous or intramuscular), and analyzed for calcium deposits or osteocalcin expression (Northem blot).

All references cited herein are incorporated herein by reference in their entirety and or all purposes to the same extent as if each individual publication, patent or patent pplication was specifically and individually indicated to be incorporated by reference in its ntirety for all purposes.

The citation of any publication is for its disclosure prior to the filing date and should not be construed as an admission that the present invention is not entitled to antedate such publication by virtue of prior invention.

Many modifications and variations of this invention can be made without departing from its spirit and scope, as will be apparent to those skilled in the art. The specific embodiments exemplified herein are offered by way of example only, and the invention is to be limited only by the terms of the appended claims along with the full scope of equivalents to which such claims are entitled.

What is claimed is:

1. A method of genetically transforming a vertebrate microvascular free flap with a nucleic acid encoding a product of interest comprising:
(a) transfecting cells of the free flap ex vivo by perfusing the free flap with a suspension comprising a vector wherein the vector comprises the nucleic acid encoding the product of interest under the control of a promoter; and
(b) reattaching the free flap to the native circulation using microvascular surgical techniques.

2. A method of genetically transforming a selected tissue of a vertebrate microvascular free flap with a nucleic acid encoding a product of interest comprising:
(a) detaching a microvascular flap from the native circulation;
(b) transfecting cells of said free flap ex vivo by perfusing the free flap with a suspension comprising a vector wherein the vector comprises the nucleic acid encoding the product of interest under the control of a promoter; and
(c) reattaching the free flap to the native circulation. using microvascular surgical techniques.

3. The method of claim 1 or 2 wherein the vertebrate free flap is mammalian.

4. The method of claim 1 or 2 wherein the vertebrate free flap is human.

5. The method of claim 1 or 2 wherein the vector is an adenoviral vector.

6. The method of claim 1 or 2 wherein the concentration of the suspension is 2.5×10⁹ to 4×10¹¹ PFU/ml.

7. The method of claim 1 or 2 wherein the vector is an adeno-associated viral (AAV) vector.

8. The method of claim 1 or 2 wherein the vector is a liposome.

9. The method of claim 1 or 2 wherein the promoter is CMV.

10. The method of claim 1 or 2 wherein the promoter is a tetracycline-inducible promoter system.

11. The method of claim 1 or 2 wherein the product of interest is proinsulin or insulin.

12. The method of claim 1 or 2 wherein the product of interest is endostatin.

13. The method of claim 1 or 2 wherein the product of interest is IL-12.

14. A method of modifying a vertebrate microvascular free flap comprising:
(a) introducing cells into said free flap ex vivo by perfusing the free flap with a suspension comprising the cells; and
(b) maintaining the free flap under suitable maintenance conditions for a period of time ex vivo,
wherein, after step (b), the introduced cells produce a product of interest.

15. A method of modifying a vertebrate microvascular free flap comprising:
(a) detaching a microvascular flap from the native circulation;
(b) introducing cells into said free flap ex vivo by perfusing the free flap with a suspension comprising the cells; and
(c) maintaining the. free flap under suitable maintenance conditions for a period of time ex vivo,
wherein, after step (c), the introduced cells produce a product of interest.

16. The method of claim 14 further comprising
(c) reattaching the free flap to the native circulation using microvascular surgical techniques.

17. The method of claim 15 further comprising
(d) reattaching the free flap to the native circulation using microvascular surgical techniques.

18. The method of claim 14 or 15 wherein the vertebrate free flap is mammalian.

19. The method of claim 14 or 15 wherein the vertebrate free flap is human.

20. The method of claim 14 or 15 wherein the product of interest is proinsulin or insulin.

21. The method of claim 14 or 15 wherein the product of interest is endostatin.

22. The method of claim 14 or 15 wherein the product of interest is IL-12.

23. The method of claim 14 wherein the period of time in step (b) is two weeks.

24. The method of claim 14 wherein the period of time in step (b) is three weeks.

25. The method of claim 15 wherein the period of time in step (c) is two weeks.

26. The method of claim 15 wherein the period of time in step (c) is three weeks.

27. The method of claim 14 or 15 wherein suitable maintenance conditions omprise maintenance in a bioreactor.

28. The method of claim 14 or 15 wherein the cells are stem cells.

29. The method of claim 14 or 15 wherein the cells are derived from stem cells differentiated ex vivo.

30. The method of claim 14 or 15 wherein the cells are genetically transformed cells.

31. A genetically transformed vertebrate microvascular free flap comprising a detached microvascular flap transfected with a vector which comprises a nucleic acid encoding a product of interest under the control of a promoter, said free flap suitable for implantation into a vertebrate host.

32. The flap of claim 31 that is reattached to the native circulation of the host.

33. The flap of claim 31 wherein the vertebrate microvascular free flap is mammalian.

34. The flap of claim 31 wherein the vertebrate microvascular free flap is human.

35. The flap of claim 31 wherein the vector is an adenoviral vector.

36. The flap of claim 31 wherein the concentration of the suspension is 2.5×10⁹ to 4×10¹¹ PFU/ml.

37. The flap of claim 31 wherein the vector is an adeno-associated viral (AAV) ector.

38. The flap of claim 31 wherein the vector is a liposome.

39. The flap of claim 31 wherein the promoter is CMV.

40. The flap of claim 31 wherein the promoter is a tetracycline-inducible promoter system.

41. The flap of claim 31 wherein the product of interest is proinsulin or insulin.

42. The flap of claim 31 wherein the product of interest is endostatin.

43. The flap of claim 31 wherein the product of interest is IL-12.

44. A modified vertebrate rnicrovascular free flap comprising a detached microvascular flap comprising cells that have been introduced into the flap ex vivo, wherein said introduced cells produce a product of interest and said free flap is suitable for implantation into a vertebrate host.

45. The flap of claim 44 that is genetically transformed ex vivo.

46. The flap of claim 44 that is reattached to the native circulation.

47. The flap of claim 44 wherein the vertebrate microvascular free flap is mammalian.

48. The flap of claim 44 wherein the vertebrate microvascular free flap is human.

49. The flap of claim 44 wherein the product of interest is proinsulin or insulin.

50. The flap of claim 44 wherein the product of interest is endostatin.

51. The flap of claim 44 wherein the product of interest is IL-12.

52. The flap of claim 44 wherein the cells are stem cells.

53. The flap of claim 44 wherein the cells are derived from stem cells differentiated ex vivo.

54. The flap of claim 44 wherein the cells are genetically transformed cells.

55. The flap of claim 44 wherein the flap is maintained under suitable maintenance conditions for a period of time ex vivo.

56. The flap of claim 55 wherein the period of time is two weeks.

57. The flap of claim 55 wherein the period of time is three weeks.

58. The flap of claim 55 wherein suitable maintenance conditions comprise maintenance in a bioreactor.

59. The method of claim 1 or claim 2 wherein the reattached free flap produces a product of interest.

60. The flap of claim 31 that produces a product of interest.


SEQUENCE LISTING (2 sequences)

SEQ ID NO: 1 - DNA, 22 bp, Artificial
  Description of Artificial Sequence Primer
gatcaaatctgtcgatccttcc

SEQ ID NO: 2 - DNA, 22 bp, Artificial
  Description of Artificial Sequence Primer
caaagaccagaccgttcataca